(19) United States
(12) Patent Application Publication
(10) Pub. No.: US 2009/0082354 A1
(43) Pub. Date: Mar. 26, 2009

(54) PYRAZOLO[5, 1-C] [1,2,4] TRIAZINES, METHODS FOR PREPARATION AND USE THEREOF

(75) Inventors: Dan Maarten Berger, New City, NY (US); Minu Dhanjisha Dutia, West Nyack, NY (US); Darrin William Hopper, New York, NY (US); Nancy Torres, Nyack, NY (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/233,877

(22) Filed: Sep. 19, 2008

Related U.S. Application Data
(60) Provisional application No. 60/994,589, filed on Sep. 20, 2007.

(51) Int. Cl. A61K 31/53; C07D 487/04

(52) U.S. Cl. 514/243; 544/184

(57) ABSTRACT

A pyrazolo[5,1-c][1,2,4]triazine compound of formula A:

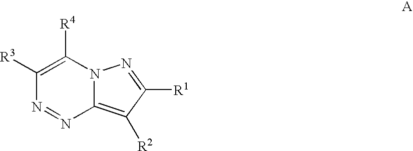

and pharmaceutically acceptable sats thereof. The multiply-substituted pyrazolo[5,1-c][1,2,4]triazine compounds of the invention selectively inhibit B-Raf kinase activity and are useful for treating disorders mediated by B-Raf kinase.

### CROSS-REFERENCE TO RELATED APPLICATION

This application claims priority benefit of U.S. Provisional Application Ser. No. 60/994,589 filed Sep. 20, 2007, which is incorporated herein by reference in its entirety.

### FIELD OF THE INVENTION

The present invention relates to new heterocyclic compositions that are useful for inhibiting abnormal growth of certain cell types. The invention is directed to certain substituted pyrazolo[5,1-c][1,2,4]triazines, their corresponding pharmaceutically acceptable salts and methods for their preparation and use. The substituted pyrazolo[5,1-c][1,2,4]triazines inhibit growth of tumor cells, which contain oncogenic forms of Receptor Tyrosine Kinases, K-Ras and B-Raf kinase.

### BACKGROUND OF THE INVENTION

B-Raf kinase is one of three known Raf oncoprotein kinases involved in transmission of mitogenic and anti-apoptotic signals. B-Raf encodes a Ras-regulated kinase that mediates cell growth and malignant transformation pathway activation that controls cell growth and survival. Activation of a Ras/Raf/MEK pathway results in a cascade of events from the cell surface to the cell nucleus, ultimately affecting cell proliferation, apoptosis, differentiation and transformation. Activating B-Raf mutations have been found in 66% of malignant melanomas and in a smaller fraction of other cancers including those of the colorectum, as reported by Davies H., et al. (2002) Nature 417:906 and by Rajagopalan H., et al. (2002) Nature 418:934. Recently, B-raf has been shown to be frequently mutated in various human cancers, as described by Wan et al. (2004) Cell 116:855-867. Therefore, it is desirable to identify and characterize compounds that inhibit growth of tumor cells, which contain oncogenic forms of Receptor Tyrosine Kinases, K-Ras and B-Raf kinase. U.S. Pat. No. 5,478,827 describes a few examples of simple tetrahydropyrazolo[5,1-c][1,2,4]triazines, which are disclosed as inhibitors of interleukin-1 and tumor necrosis factor, associated with chronic inflammation diseases (e.g. rheumatoid arthritis and osteoarthritis). However, no structure-activity relationships (SAR) have been described for the pyrazolo[5,1-c][1,2,4]triazines and little is known regarding how various functional groups substituted at multiple positions of the fused heterocycle ring framework influence the SAR of pyrazolo[5,1-c][1,2,4]triazines. There is a need for new compounds that selectively inhibit B-Raf kinase activity and that are useful for treating disorders mediated by B-Raf kinase. Multiply-substituted pyrazolo[5,1-c][1,2,4]triazine compositions of the present invention fulfill this unmet need and are useful in the treatment of cancer including, but not limited to for example, colonic polyps, in mammals.

### SUMMARY OF THE INVENTION

Accordingly, the invention provides a compound of formula A:

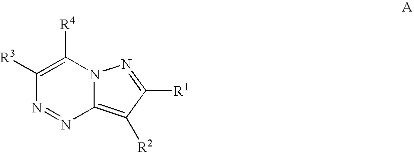

and pharmaceutically acceptable sats thereof;
wherein R¹ is a 5-7 membered heterocyclic ring or heteroaryl ring containing 1-3 heteroatoms selected from N, O or S, or an aryl ring, each ring substituted with one to four substituents selected from the group consisting of: —F, —Cl, Br, —I, —NO₂, —CN, —N₃, —CHO, —CF₃, —OCF₃, —R⁵, —OR⁵, —S(O)ₘR⁵, —S(O)ₘNR⁵R⁵, —NR⁵R⁵, —NR⁵S(O)ₘR⁵, —OR⁷OR⁵, —OR⁷NR⁵R⁵, —N(R⁵)R⁷OR⁵, —N(R⁵)R⁷NR⁵R⁵, —NR⁵C(O)R⁵, —C(O)R⁵, —C(O)OR⁵, —C(O)NR⁵R⁵, —OC(O)R⁵, —OC(O)OR⁵, —OC(O)NR⁵R⁵, NR⁵C(O)R⁵, —NR⁵C(O)OR⁵, —NR⁵C(O)NR⁵R⁵, —R⁶OR⁵, —R⁶OR⁷OR⁵, —R⁶OR⁷NR⁵R⁵, —R⁶N(R⁵)R⁷OR⁵, —R⁶N(R⁵)R⁷NR⁵R⁵, —R⁶NR⁵R⁵, —R⁶S(O)ₘR⁵, —R⁶S(O)ₘNR⁵R⁵, —R⁶C(O)R⁵, —R⁶C(O)R⁵, —R⁶C(O)NR⁵R⁵, —R⁶OC(O)R⁵, —R⁶OC(O)OR⁵, —R⁶NR⁵S(O)ₘR⁵, —R⁶OC(O)NR⁵R⁵, —R⁶NR⁵C(O)R⁵, —R⁶NR⁵C(O)OR⁵ or —R⁶NR⁵C(O)NR⁵R⁵;
R² is an aryl ring substituted with at least one substituent —OR⁸, up to four other substituents, each other substituent independently selected from the group consisting of: —F, —Cl, Br, —I, —NO₂, —CN, —N₃, —CHO, —CF₃, —OCF₃, —R⁵, —OR⁵, —S(O)ₘR⁵, —NR⁵R⁵, —NR⁵S(O)ₘR⁵, —S(O)ₘNR⁵R⁵, —OR⁷OR⁵, —OR⁷NR⁵R⁵, —N(R⁵)R⁷OR⁵, —N(R⁵)R⁷NR⁵R⁵, —NR⁵C(O)R⁵, —C(O)R⁵, —C(O)OR⁵, —C(O)NR⁵R⁵, —OC(O)R⁵, —OC(O)OR⁵, —OC(O)NR⁵R⁵, NR⁵C(O)R⁵, —NR⁵C(O)OR⁵, —NR⁵C(O)NR⁵R⁵, —R⁶OR⁵, —R⁶OR⁷OR⁵, —R⁶OR⁷NR⁵R⁵, —R⁶N(R⁵)R⁷OR⁵, —R⁶N(R⁵)R⁷NR⁵R⁵, —R⁶NR⁵R⁵, —R⁶S(O)ₘR⁵, —R⁶NR⁵S(O)ₘR⁵, —R⁶S(O)ₘNR⁵R⁵, —R⁶C(O)R⁵, —R⁶C(O)OR⁵, —R⁶C(O)NR⁵R⁵, —R⁶OC(O)R⁵, —R⁶OC(O)OR⁵, —R⁶OC(O)NR⁵R⁵, —R⁶NR⁵C(O)R⁵, —R⁶NR⁵C(O)OR⁵ or —R⁶NR⁵C(O)NR⁵R⁵;
R³ and R⁴ are independently selected from the group consisting of: H, cycloalkyl of 3-10 carbons, alkyl of 1-6 carbons, alkoxy of 1-6 carbons, cycloalkoxy of 3-10 carbons, alkene of 1-6 carbons, alkyne of 1-6 carbons; aryl ring, heterocyclic ring and heteroaryl ring containing 1-3 heteroatoms selected from N, O or S; each ring substituted with one to four substituents selected from the group consisting of: —F, —Cl, Br, —I, —NO₂, —CN, —N3, —CHO, —OCF₃, —S(O)ₘR⁸, —NR⁸R⁸, —NR⁸S(O)ₘR⁸, —S(O)ₘNR⁸R⁸, —OR⁷OR⁸, —OR⁷NR⁸R⁸, —N(R⁸)R⁷OR⁸, —N(R⁸)R⁷NR⁸R⁸, —NR⁸C(O)R⁸, —C(O)R⁸, —C(O)OR⁸, —C(O)NR⁸R⁸, —OC(O)R⁸, —OC(O)OR⁸, —OC(O)NR⁸R⁸, NR⁸C(O)R⁸, —NR⁸C(O)OR⁸, —NR⁸C(O)NR⁸R⁸, —R⁶OR⁸, —R⁶NR⁸R⁸, —R⁶S(O)ₘR⁸, —R⁶NR⁸S(O)ₘR⁸, —R⁶S(O)ₘNR⁸R⁸, —R⁶C(O)R⁸, —R⁶C(O)OR⁸, —R⁶OR⁷OR⁸, —R⁶OR⁷NR⁸R⁸, —R⁶N(R⁸)R⁷OR⁸, —R⁶N(R⁸)R⁷NR⁸R⁸, —R⁶C(O)NR⁸R⁸, —R⁶OC(O)R⁸, —R⁶OC(O)OR⁸, —R⁶OC(O)NR⁸R⁸, —R⁶NR⁸C(O)R⁸, —R⁶NR⁸C(O)OR⁸, —R⁶NR⁸C(O)NR⁸R⁸ or —YR⁸;
R⁵ is selected from the group consisting of: H, alkyl of 1-6 carbon atoms, branched alkyl of 1-8 carbon atoms, alkenyl of 2-6 carbon atoms, alkynyl of 2-6 carbon atoms or cycloalkyl of 3-7 carbons;
R⁶ is a divalent group selected from the group consisting of: alkyl of 1-6 carbon atoms, alkenyl of 2-6 carbon atoms, and alkynyl of 2-6 carbon atoms;
R⁷ is a divalent alkyl group of 2-6 carbon atoms;
R⁸ is selected from the group consisting of: H, alkyl of 1-6 carbon atoms, alkenyl of 2-6 carbon atoms, alkynyl of 2-6 carbon atoms, aryl of 6-12 carbons, substituted aryl of 6-12 carbons, substituted heterocyclyl ring and substituted heteroaryl ring containing 1-3 heteroatoms selected from N, O or S;
m is an integer of 0-2; and

### Y is —N(R⁵)—, —N(R⁵)R⁶—, —O—, —OR⁶—, —S(O)ₘ— or —S(O)ₘR⁶.

The present invention also provides a compound of formula A and pharmaceutically acceptable sats thereof; wherein R² is a bicyclic heteroaryl ring of formula

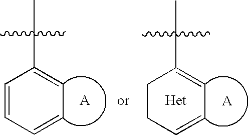

wherein

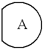

is a 5-7 membered heteroaryl ring containing 1-3 heteroatoms selected from N, O or S,
Het is a 6-membered heteroaryl ring containing 1-2 nitrogen atoms, and either bicyclic heteroaryl ring is substituted with one to four substituents, each substituent independently selected from the group consisting of: —F, —Cl, Br, —I, —NO₂, —CN, —N₃, —CHO, —CF₃, —OCF₃, —R⁵, —OR⁵, —S(O)ₘR⁵, —NR⁵R⁵, with R¹⁻⁸ as defined above.

The present invention also provides a pharmaceutical composition comprising a compound of formula A and a pharmaceutically acceptable carrier. The present invention also provides pharmaceutical compositions comprising compounds of formula A alone or in combination with other kinase-inhibiting pharmaceutical compositions or chemotherapeutic agents, and a pharmaceutically acceptable carrier.

The present invention provides a method for making a compound of formula A:

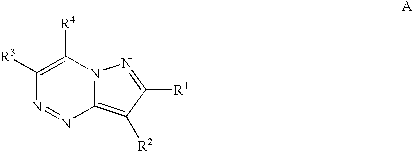

and pharmaceutically acceptable sats thereof; comprising the steps of: (a) reacting a substituted aminopyrazole of formula

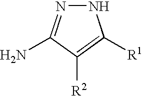

with a mixture of sodium nitrate and a strong acid in an aqueous solvent; and (b) adding a substituted β-keto acid or a substituted β-keto ester of formula:

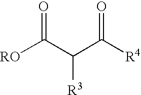

and a base to the mixture, wherein R is selected from: H, an alkyl of 1-6 carbon atoms, an aryl of 6-12 carbons and a substituted aryl of 6-12 carbons, and R¹⁻⁴ are as defined above.

The present invention also provides a method for making a compound of formula A:

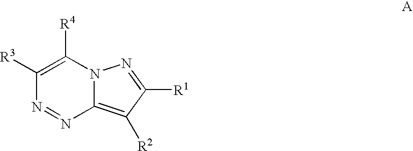

and pharmaceutically acceptable sats thereof; comprising the steps of: (a) reacting a substituted aminopyrazole of formula

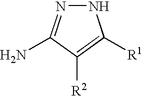

with a mixture of sodium nitrate and a strong acid in an aqueous solvent; (b) adding a substituted β-keto ester of formula:

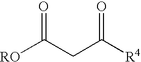

and a base to the mixture; and (c) hydrolyzng the ester formed of formula:

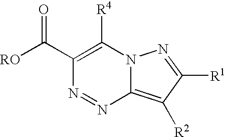

to a corresponding carboxylic acid using an aqueous acid or to a corresponding carboxylate salt using an aqueous base; and (d) heating the compound of formula:

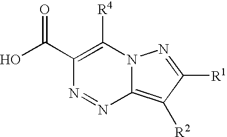

to a temperature of 150° C. or higher, wherein R and R¹⁻⁴ are as defined above.

The present invention also provides a method for making a compound of formula A comprising the step of reacting the compound of formula:

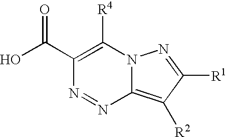

with substituted amines, wherein R and R¹⁻⁴ are as defined above.

The present invention also provides a method for making a compound of formula A:

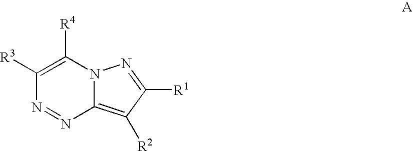

and pharmaceutically acceptable sats thereof; comprising the steps of: (a) reacting a substituted aminopyrazole of formula

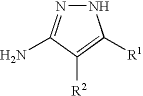

with a mixture of sodium nitrate and a strong acid in an aqueous solvent; (b) adding a substituted β-keto sulfone of formula:

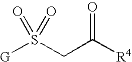

and a base to the mixture; and (c) reducing the sulfone formed of formula:

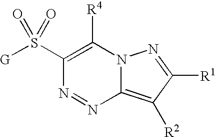

with a reducing agent, wherein G is selected from an alkyl of 1-6 carbon atoms, an aryl of 6-12 carbons and a substituted aryl of 6-12 carbons, and R¹⁻⁴ are as defined above.

The invention also provides methods for inhibiting B-Raf kinase activity in a cell comprising contacting a cell with a compound of formula A, whereby the compound inhibits B-raf kinase activity.

The present invention also provides a method of treating a B-Raf kinase-dependent condition, especially inflammation or cancer, by administering to a patient a compound of formula A.

The present invention provides methods of treating mammalian diseases associated with B-Raf kinase by administering to a patient a compound of formula A.

The present invention provides methods of treating cancer selected from the group consisting of: breast, kidney, bladder, mouth, larynx, esophagus, stomach, colon, ovary, lung, pancreas, skin, liver, prostate and brain cancer.

### DETAILED DESCRIPTION OF THE INVENTION

### Definitions

The following definitions are used in connection with pyrazolo[5,1-c][1,2,4]triazines of the invention. Unless otherwise defined, the term “aryl”, as used herein, refers to an aromatic carbocyclic moiety, e.g. having from 6-20 carbon atoms, which may be a single ring (monocyclic) or multiple rings fused together or linked covalently, wherein at least one of the rings is aromatic. Any suitable ring position of the aryl moiety may be covalently linked to the defined chemical structure. Examples of aryl include phenyl and napthyl. The aryl group may be optionally substituted. In addition to other optional substituents, the aryl group may be substituted by an oxo substituent meaning one of the ring carbon atoms is part of a carbonyl group.

Unless otherwise defined, the term “heteroaryl” as used herein means an aromatic heterocyclic ring system, e.g. having from 5-20 ring atoms, which may be a single ring or multiple rings fused together or linked covalently, wherein at least one of the rings is aromatic. The rings may contain one or more heteroatoms, e.g. 1 to 3 heteroatoms, selected from nitrogen, oxygen, or sulfur, wherein the nitrogen or sulfur atom(s) are optionally oxidized, or the nitrogen atom(s) are optionally quaternized. Any suitable ring position of the heteroaryl moiety may be covalently linked to the defined chemical structure. Examples of heteroaryl include 2-pyridyl or indol-1-yl. The heteroaryl group may be optionally substituted. In addition to other optional substituents, the heteroaryl group may be substituted by an oxo substituent meaning one of the ring carbon atoms is part of a carbonyl group.

The term “heterocyclic”, “heterocycle” or “heterocyclyl” as used herein can be used interchangeably to refer to a stable, saturated or partially unsaturated monocyclic or multicyclic heterocyclic ring system, including a spirocyclic and bridged heterocyclic ring system, e.g. having from 5 to 7 ring members. The heterocyclic ring members are carbon atoms and one or more heteroatoms, e.g. 1 to 3 heteroatoms, selected from nitrogen, oxygen, and sulfur atoms, wherein the nitrogen or sulfur atom(s) are optionally oxidized, or the nitrogen atom(s) are optionally quaternized. The heterocyclic, heterocycle or heterocyclyl group may be optionally substituted. In addition to other optional substituents, the heterocyclic, heterocycle or heterocyclyl group may be substituted by an oxo substituent meaning one of the ring carbon atoms is part of a carbonyl group. The heterocyclic, heterocycle or heterocyclyl group may contain one of more fused rings.

The term “bicyclic heteroaryl ring” refers to a ring framework of formula

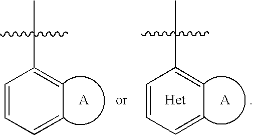

The symbol

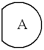

refers to a 5-7 membered heteroaryl ring containing 1-3 heteroatoms selected from N, O or S. The term “Het” refers to a 6-membered heteroaryl ring containing 1-2 nitrogen atoms. Either bridged bicyclic heteroaryl ring is substituted with one to four substituents, each substituent independently selected from the group consisting of: —F, —Cl, Br, —I, —NO₂, —CN, —N₃, —CHO, —CF₃, —OCF₃, —R⁵, —OR⁵, —S(O)ₘR⁵, —NR⁵R⁵.

As used herein, the term “pharmaceutically acceptable carrier” includes pharmaceutically acceptable diluents and excipients.

As used herein, the term “individual”, “subject” or “patient,” used interchangeably, refers to any animal, including mammals, preferably mice, rats, other rodents, rabbits, dogs, cats, swine, cattle, sheep, horses, or primates, and most preferably humans.

According to an exemplary embodiment, the invention provides a compound of formula A:

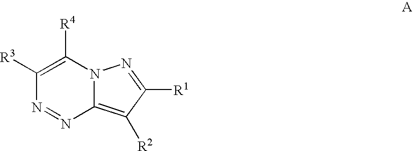

and pharmaceutically acceptable sats thereof;
wherein R¹ is a 5-7 membered heterocyclic ring or a heteroaryl ring containing 1-3 heteroatoms selected from N, O or S, or an aryl ring, each ring substituted with one to four substituents selected from the group consisting of: —F, —Cl, Br, —I, —NO₂, —CN, —N₃, —CHO, —CF₃, —OCF₃, —R⁵, —OR⁵, —S(O)ₘR⁵, —S(O)ₘNR⁵R⁵, —NR⁵R⁵, —NR⁵S(O)ₘR⁵, —OR⁷OR⁵, —OR⁷NR⁵R⁵, —N(R⁵)R⁷OR⁵, —N(R⁵)R⁷NR⁵R⁵, —NR⁵C(O)R⁵, —C(O)R⁵, —C(O)OR⁵, —C(O)NR⁵R⁵, —OC(O)R⁵, —OC(O)OR⁵, —OC(O)NR⁵R⁵, NR⁵C(O)R⁵, —NR⁵C(O)OR⁵, —NR⁵C(O)NR⁵R⁵, —R⁶OR⁵, —R⁶OR⁷OR⁵, —R⁶OR⁷NR⁵R⁵, —R⁶N(R⁵)R⁷OR⁵, —R⁶N(R⁵)R⁷NR⁵R⁵, —R⁶NR⁵R⁵, —R⁶S(O)ₘR⁵, —R⁶S(O)ₘNR⁵R⁵, —R⁶C(O)R⁵, —R⁶C(O)R⁵, —R⁶C(O)NR⁵R⁵, —R⁶OC(O)R⁵, —R⁶OC(O)OR⁵, —R⁶NR⁵S(O)ₘR⁵, —R⁶OC(O)NR⁵R⁵, —R⁶NR⁵C(O)R⁵, —R⁶NR⁵C(O)OR⁵ or —R⁶NR⁵C(O)NR⁵R⁵;
R² is a monocyclic aryl ring substituted with at least one substituent —OR⁸, up to four other substituents, each other substituent independently selected from the group consisting of: —F, —Cl, Br, —I, —NO₂, —CN, —N₃, —CHO, —CF₃, —OCF₃, —R⁵, —OR⁵, —S(O)ₘR⁵—NR⁵R⁵, —NR⁵S(O)ₘR⁵, —S(O)ₘNR⁵R⁵, —OR⁷OR⁵, —OR⁷NR⁵R⁵, —N(R⁵)R⁷OR⁵, —N(R⁵)R⁷NR⁵R⁵, —NR⁵C(O)R⁵, —C(O)R⁵, —C(O)OR⁵, —C(O)NR⁵R⁵, —OC(O)R⁵, —OC(O)OR⁵, —OC(O)NR⁵R⁵, NR⁵C(O)R⁵, —NR⁵C(O)OR⁵, —NR⁵C(O)NR⁵R⁵, —R⁶OR⁵, —R⁶OR⁷OR⁵, —R⁶OR⁷NR⁵R⁵, —R⁶N(R⁵)R⁷OR⁵, —R⁶N(R⁵)R⁷NR⁵R⁵, —R⁶NR⁵R⁵, —R⁶S(O)ₘR⁵, —R⁶NR⁵S(O)ₘR⁵, —R⁶S(O)ₘNR⁵R⁵, —R⁶C(O)R⁵, —R⁶C(O)OR⁵, —R⁶C(O)NR⁵R⁵, —R⁶OC(O)R⁵, —R⁶OC(O)OR⁵, —R⁶OC(O)NR⁵R⁵, —R⁶NR⁵C(O)R⁵, —R⁶NR⁵C(O)OR⁵ or —R⁶NR⁵C(O)NR⁵R⁵;

R³ and R⁴ are independently selected from the group consisting of: H, cycloalkyl of 3-10 carbons, alkyl of 1-6 carbons, alkoxy of 1-6 carbons, cycloalkoxy of 3-10 carbons, alkene of 1-6 carbons, alkyne of 1-6 carbons; aryl ring, heterocyclic ring and heteroaryl ring containing 1-3 heteroatoms selected from N, O or S; each ring substituted with one to four substituents selected from the group consisting of: —F, —Cl, Br, —I, —NO₂, —CN, —N₃, —CHO, —OCF₃, —S(O)ₘR⁸, —NR⁸R⁸, —NR⁸S(O)ₘR⁸, —S(O)ₘNR⁸R⁸, —OR⁷OR⁸, —OR⁷NR⁸R⁸, —N(R⁸)R⁷OR⁸, —N(R⁸)R⁷NR⁸R⁸, —NR⁸C(O)R⁸, —C(O)R⁸, —C(O)OR⁸, —C(O)NR⁸R⁸, —OC(O)R⁸, —OC(O)OR⁸, —OC(O)NR⁸R⁸, NR⁸C(O)R⁸, —NR⁸C(O)OR⁸, —NR⁸C(O)NR⁸R⁸, —R⁶OR⁸, —R⁶NR⁸R⁸, —R⁶S(O)ₘR⁸, —R⁶NR⁸S(O)ₘR⁸, —R⁶S(O)ₘNR⁸R⁸, —R⁶C(O)R⁸, —R⁶C(O)OR⁸, —R⁶OR⁷OR⁸, —R⁶OR⁷NR⁸R⁸, —R⁶N(R⁸)R⁷OR⁸, —R⁶N(R⁸)R⁷NR⁸R⁸, —R⁶C(O)NR⁸R⁸, —R⁶OC(O)R⁸, —R⁶OC(O)OR⁸, —R⁶OC(O)NR⁸R⁸, —R⁶NR⁸C(O)R⁸, —R⁶NR⁸C(O)OR⁸, —R⁶NR⁸C(O)NR⁸R⁸ or —YR⁸;

R⁵ is selected from the group consisting of: H, alkyl of 1-6 carbon atoms, branched alkyl of 1-8 carbon atoms, alkenyl of 2-6 carbon atoms, alkynyl of 2-6 carbon atoms or cycloalkyl of 3-7 carbons;
R⁶ is a divalent group selected from the group consisting of: alkyl of 1-6 carbon atoms, alkenyl of 2-6 carbon atoms, and alkynyl of 2-6 carbon atoms;
R⁷ is a divalent alkyl group of 2-6 carbon atoms;
R⁸ is selected from the group consisting of: H, alkyl of 1-6 carbon atoms, alkenyl of 2-6 carbon atoms, alkynyl of 2-6 carbon atoms, aryl of 6-12 carbons, substituted aryl of 6-12 carbons, substituted heterocyclyl ring and substituted heteroaryl ring containing 1-3 heteroatoms selected from N, O or S;
m is an integer of 0-2; and

### Y is —N(R⁵)—, —N(R⁵)R⁶—, —O—, —OR⁶—, —S(O)ₘ— or —S(O)ₘR⁶.

Suitable examples of R¹ include, but are not limited to for example, thienyl, furyl, indolyl, pyrrolyl, thiophenyl, benzofuryl, benzothiophenyl, quinolyl, isoquinolyl, imidazolyl, thiazolyl, oxazolyl, pyridinyl, pyrrolidyl, oxolanyl, thiolanyl, piperidinyl, piperazinyl, thiazolyl, triazolyl, pyrazolyl, pyrazinyl, pyridazinyl, pyrimidinyl, and morpholinyl. The heterocyclic ring or heteroaryl ring may be substituted to the pyrazolo[5,1-c][1,2,4]triazine ring framework in any acceptable position. According to one embodiment, R¹ is selected from the group consisting of: 2-pyridinyl, 3-pyridinyl, 4-pyridinyl, 5-pyridinyl, 6-pyridinyl, 2-piperidinyl, 3-piperidinyl, 4-piperidinyl, 5-piperidinyl, 6-piperidinyl, indol-1yl, indol-2-yl and indol-3yl.

According to one embodiment, R² is selected from the group consisting of: phenols, halogen substituted phenols, alkoxy substituted phenyls, phenoxy substituted phenyls, benzyloxy substituted phenyls, methoxy substituted phenyl and phenyl substituted with alkoxy and halogen. The monocyclic aryl ring may be substituted to the pyrazolo[5,1-c][1,2,4]triazine ring framework in any acceptable position. According to one embodiment, R² is selected from the group consisting of: 2-phenol, 3-phenol, 4-phenol, 4-chloro-3-phenol, 3-chloro-4-phenol, 3-chloro-2-phenol, 2-methoxyphenyl, 3-methoxyphenyl, 4-methoxyphenyl, 4-chloro-3-methoxyphenyl, 3-chloro-4-methoxyphenyl, 3-chloro-2-methoxyphenyl, 2-(benzyloxyl)phenyl, 3-(benzyloxyl)phenyl and 4-(benzyloxyl)phenyl.

According to a separate embodiment, R² is a bicyclic heteroaryl ring of formula

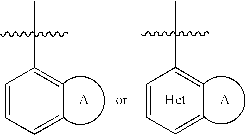

wherein

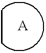

is a 5-7 membered heteroaryl ring containing 1-3 heteroatoms selected from N, O or S,
Het is a 6-membered heteroaryl ring containing 1-2 nitrogen atoms, and either bridged bicyclic heteroaryl ring is substituted with one to four substituents, each substituent independently selected from the group consisting of: —F, —Cl, Br, —I, —NO₂, —CN, —N₃, —CHO, —CF₃, —OCF₃, —R⁵, —OR⁵, —S(O)ₘR⁵, —NR⁵R⁵, with R¹⁻⁸ as defined above. Suitable examples of R² rings include, but are not limited to, indolyl, benzimidazolyl, indazolidinyl, benzotriazolyl, oxindolyl, benzothiazolonyl and benzooxazolonyl.

According to one embodiment, R³ is selected from the group consisting of: H—, C₁-C₆ alkyl esters, C₁-C₆ alkyl amino, C₁-C₆ alkyl amido, C₁-C₆ substituted heterocyclic amino, C₁-C₆ substituted heterocyclic amido, C₁-C₆ alkyl sulfonyl, aryl sulfonyl and heteroaryl sulfonyl.

According to one embodiment, R⁴ is selected from the group consisting of: C₁-C₆ alkyl, C₃-C₈ cycloalkyl, heteroaryl, substituted heteroaryl, halogen substituted pyridinyl, heterocyclic substituted pyridinyl, diazepanyl substituted pyridinyl, piperazinyl substituted pyridinyl, substituted phenyl, heteroarylsulfonyl substituted phenyl, hetrocyclosulfonyl substituted aryl, hetrocyclosulfonyl substituted phenyl, bicycloalkyl and heterobicycloalkyl. The heteroaryl, aryl, bicycloalkyl and heterobicycloalkyl rings may be substituted to the pyrazolo[5,1-c][1,2,4]triazine ring framework in any acceptable position.

The compounds of this invention may be prepared from: (a) commercially available starting materials (b) known starting materials which may be prepared as described in literature procedures or (c) new intermediates described in the schemes and experimental procedures herein.

Reactions are performed in a solvent appropriate to the reagents and materials employed and suitable for the transformation being effected. It is understood by those skilled in the art of organic synthesis that the various functionalities present on the molecule must be consistent with the chemical transformation proposed. This may necessitate judgement as to the order of synthetic steps.

Compounds of the present invention may be prepared as illustrated in the examples and in following reaction schemes 1 to 4.

Scheme 1 outlines the synthesis of certain substituted aminopyrazole intermediates:

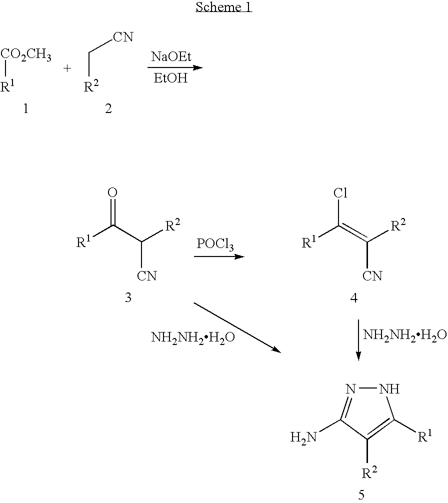

Referring to Scheme 1, the condensation reaction of substituted acetonitriles 2 with substituted esters 1 can be carried out in the presence of a base such as, but not limited to sodium ethoxide, in a suitable solvent such as ethanol to provide intermediates 3. Intermediates 3 can subsequently be reacted with hydrazine hydrate in a suitable solvent such as ethanol to provide aminopyrazoles 5 where R¹ and R² are herein before defined. For certain substituted intermediates 3, it is necessary to first react with phosphorus oxychloride at elevated temperatures, typically at reflux, to provide intermediates 4. Intermediates 4 can be converted to substituted aminopyrazoles 5 by subsequent reaction with hydrazine hydrate in a suitable solvent such as ethanol. Substituted esters 1 and substituted acetonitriles 2 can be obtained from commercial sources or readily prepared by numerous literature procedures by those skilled in the art.

The reaction of intermediates 5 with sodium nitrite in the presence of a strong acid such as hydrochloric or sulfuric acid in a protic solvent such as water and water/methanol or water/ethanol, followed by the addition of substituted β keto acids or esters 6 (R=hydrogen, alkyl of 1-6 carbon atoms, an aryl of 6-12 carbons and a substituted aryl of 6-12 carbons) and a neutralizing base such as, but not limited to sodium acetate, provides target compounds 7, where R¹, R², R³ and R⁴ are herein before defined (Scheme 2).

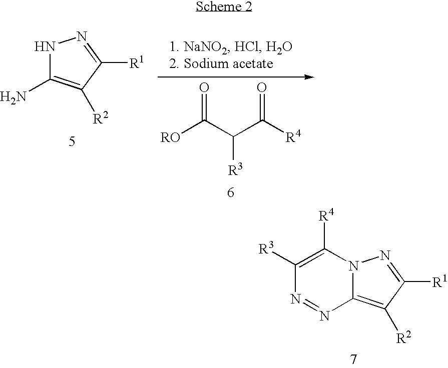

Additional preparations of the compounds of Formula A of the present invention are described below in Scheme 3 where R¹, R², R³ and R⁴ are as defined herein before. The reaction of intermediates 5 with sodium nitrite in the presence of a strong acid such as hydrochloric or sulfuric acid in a protic solvent such as water and water/methanol or water/ethanol, followed by the addition of substituted β-keto esters 6 (R=H, alkyl of 1-6 carbon atoms, an aryl of 6-12 carbons and a substituted aryl of 6-12 carbons) and a neutralizing base such as, but not limited to sodium acetate, provides target compounds 9. The ester residue of intermediates 9 can be hydrolyzed by an aqueous acid such as, but not limited to sulfuric acid, or a strong aqueous base solution of sodium hydroxide or the like, to provide intermediates 10. Heating intermediates 10 at temperatures at or above greater than 150° C. in a suitable solvent including but not limited to, for example, mixtures of biphenyl and diphenyl oxide available as Dowtherm™, provides target compounds 11 where R¹, R² and R⁴ are as defined herein before. Alternatively, intermediates 10 can be reacted with various substituted amines R′R″NH(R′ and R″ are independently selected from H, alkyl of 1-6 carbon atoms, an aryl of 6-12 carbons and a substituted aryl of 6-12 carbons) to provide a series of amide substituted compounds 12 where R, R¹, R² and R⁴ are as defined herein before.

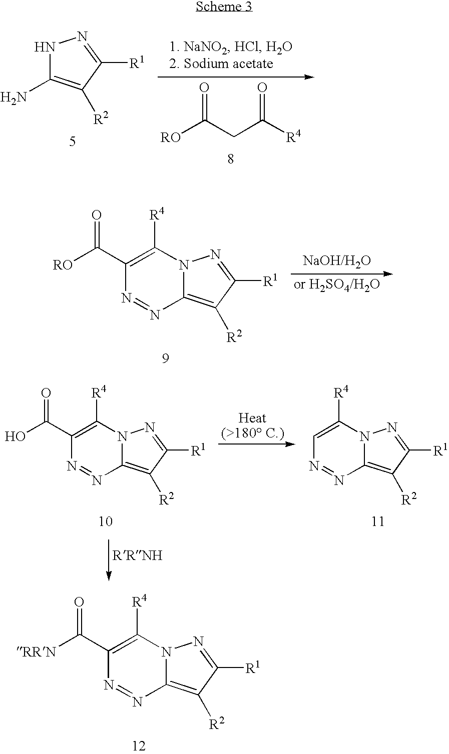

Additional preparations of the compounds of Formula A of the present invention are described below in Scheme 4 where R¹, R², R³ and R⁴ are as defined herein before. The reaction of intermediates 5 with sodium nitrite in the presence of a strong acid such as hydrochloric or sulfuric acid in a protic solvent such as water and water/methanol or water/ethanol, followed by the addition of substituted β-keto sulfones 13 (G=alkyl of 1-6 carbon atoms, an aryl of 6-12 carbons and a substituted aryl of 6-12 carbons) and a neutralizing base such as, but not limited to sodium acetate, provides compounds 14. The sulfone residue of intermediates 14 can be reduced to the corresponding sulfides by a variety of reducing agents such as triphenyl phosphine, or tributyltin hydride and the like. Reacting sulfones 14 with appropriate reducing agents, such as Raney Nickel or sodium amalgam, could provide target compounds 11 where R¹, R² and R⁴ are as defined herein before.

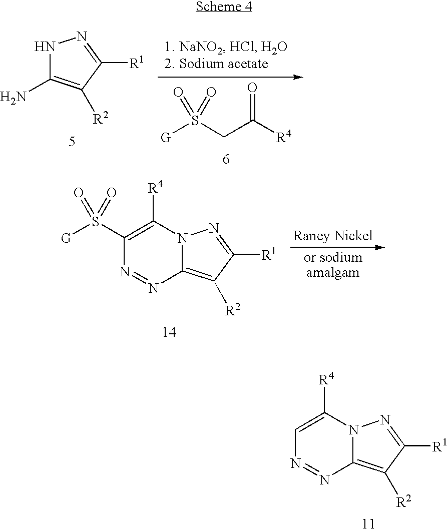

Exemplary compounds of Formula A prepared by methods of the present invention include the following compounds:

| Example | Compound Name |
|---|---|
| 1. | Ethyl 8-[3-(benzyloxy)phenyl]-4-cyclohexyl-7-pyridin-4-yl-pyrazolo[5,1- |
| | c][1,2,4]triazine-3-carboxylate |
| 2. | 3-(4-cyclohexyl-7-pyridin-4-yl-pyrazolo[5,1-c][1,2,4]triazin-8-yl)phenol |
| 3 | [8-(3-Methoxyphenyl)-4-methyl-7-pyridin-4-yl-pyrazolo[5,1-c][1,2,4]triazin3-yl-(4- |
| | methylpiperazin-1-yl)-methanone |
| 4. | 4-(6-Bromo-pyridin-3yl-)-8-(3-methoxy-phenyl)-7-pyridin-4-yl-pyrazolo[5,1- |
| | c][1,2,4]triazine-3-carboxylic acid ethyl ester |
| 5. | 3-[4-(6-Bromo-pyridin-3-yl)-7-pyridin-4-yl-pyrazolo[5,1-c][1,2,4]triazin-8-yl]-phenol |
| 6. | 3-{4-[6-(4-Methyl-[1,4]diazepan-1-yl)-pyridin-3-yl]-7-pyridin-4-yl-pyrazolo[5,1- |
| | c][1,2,4]triazin-8-yl}-phenol |
| 7. | 3-{4-[6-(4-Methyl-piperazin-1-yl)-pyridin-3-yl]-7-pyridin-4-yl-pyrazolo[5,1- |
| | c][1,2,4]triazin-8-yl}-phenol |
| 8. | 8-(3-methoxyphenyl)-4-methyl-3-(phenylsulfonyl)-7-pyridin-4-ylpyrazolo[5,1- |
| | c][1,2,4]triazine |
| 9. | 3-[4-Methyl-3-(phenylsulfonyl)-7-pyridin-4-ylpyrazolo[5,1-c][1,2,4]triazin-8- |
| | yl]phenol |
| 10. | Ethyl 8-(4-chloro-3-methoxyphenyl)-4-cyclopropyl-7-pyridin-4-ylpyrazolo[5,1- |
| | c][1,2,4]triazine-3-carboxylate |
| 11. | Ethyl 4-(6-bromopyridin-3-yl)-8-(4-chloro-3-methoxyphenyl)-7-pyridin-4- |
| | ylpyrazolo[5,1-c][1,2,4]triazine-3-carboxylate |
| 12. | 8-(4-Chloro-3-methoxy-phenyl)-4-[3-(4-methyl-piperazine-1-sulfonyl)-phenyl]-7- |
| | pyridin-4-yl-pyrazolo[5,1-c][1,2,4]triazine-3-carboxylic acid ethyl ester |
| 13. | 5-[4-(6-Bromopyridin-3-yl)-7-pyridin-4-ylpyrazolo[5,1-c][1,2,4]triazin-8-yl]-2- |
| | chlorophenol |
| 14. | 2-Chloro-5-{4-[6-(4-methyl-1,4-diazepan-1-yl)pyridin-3-yl]-7-pyridin-4- |
| | ylpyrazolo[5,1-c][1,2,4]triazin-8-yl}phenol |
| 15. | 2-Chloro-5-(4-{3-[(4-methylpiperazin-1-yl)sulfonyl]phenyl}-7-pyridin-4- |
| | ylpyrazolo[5,1-c][1,2,4]triazin-8-yl)phenol |
| 16. | Ethyl 3-[8-(4-chloro-3-methoxyphenyl)-3-(phenylsulfonyl)-7-pyridin-4- |
| | ylpyrazolo[5,1-c][1,2,4]triazin-4-yl]-8-azabicyclo[3.2.1]octane-8-carboxylate |
| 17. | Ethyl 8-(4-chloro-3-methoxyphenyl)-4-[8-(ethoxycarbonyl)-8-azabicyclo[3.2.1]oct- |
| | 3-yl]-7-pyridin-4-ylpyrazolo[5,1-c][1,2,4]triazine-3-carboxylate |
| 18. | Ethyl 3-[8-(4-chloro-3-hydroxyphenyl)-7-pyridin-4-ylpyrazolo[5,1-c][1,2,4]triazin-4- |
| | yl]-8-azabicyclo[3.2.1]octane-8-carboxylate |

The compounds of Formula A may be obtained as inorganic or organic salts using methods known to those skilled in the art, for example Richard C. Larock, Comprehensive Organic Transformations, VCH publishers, 411-415, 1989. It is well known to one skilled in the art that an appropriate salt form is chosen based on physical and chemical stability, flowability, hydroscopicity and solubility.

Pharmaceutically acceptable salts of the compounds of Formula A with an acidic moiety may be formed from organic and inorganic bases. For example with alkali metals or alkaline earth metals such as sodium, potassium, lithium, calcium, or magnesium or organic bases and N— tetraalkylammonium salts such as N-tetrabutylammonium salts. Similarly, when a compound of this invention contains a basic moiety, salts may be formed from organic and inorganic acids. For example salts may be formed from acetic, propionic, lactic, citric, tartaric, succinic, fumaric, maleic, malonic, mandelic, malic, phthalic, hydrochloric, hydrobromic, phosphoric, nitric, sulfuric, methanesulfonic, naphthalenesulfonic, benzenesulfonic, toluenesulfonic, camphorsulfonic, and similarly known acceptable acids. Suitable examples of pharmaceutically acceptable salts include, but are not limited, to sulfate; citrate, acetate; oxalate; chloride; bromide; iodide; nitrate; bisulfate; phosphate; acid phosphate; isonicotinate; lactate; salicylate; acid citrate; tartrate; oleate; tannate; pantothenate; bitartrate; ascorbate; succinate; maleate; gentisinate; fumarate; gluconate; glucaronate; saccharate; formate; benzoate; glutamate; methanesulfonate; ethanesulfonate; benzenesulfonate; p-toluenesulfonate; pamoate (i.e., 1,1′-methylene-bis-(2-hydroxy-3-naphthoate)); and salts of fatty acids such as caproate, laurate, myristate, palmitate, stearate, oleate, linoleate, and linolenate salts. The compounds can also be used in the form of esters, carbamates and other conventional prodrug forms, which when administered in such form, convert to the active moiety in-vivo.

The present invention accordingly provides a pharmaceutical composition, which comprises an effective amount of a compound of Formula A in combination or association with a pharmaceutically acceptable carrier. Pharmaceutical compositions are prepared in accordance with acceptable pharmaceutical procedures, such as described in Remingtons Pharmaceutical Sciences, 17th edition, ed. Alfonoso R. Gennaro, Mack Publishing Company, Easton, Pa. (1985). Pharmaceutically acceptable carriers are those that are compatible with the other ingredients in the formulation and biologically acceptable. As used herein, the term “effective amount” refers to the amount of active compound or pharmaceutical agent that elicits the biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease; (2) inhibiting the disease; for example, inhibiting a disease, condition or disorder in an individual that is experiencing or displaying the pathology or symptomatology of the disease, condition or disorder (i.e., arresting or slowing further development of the pathology and/or symptomatology); and (3) ameliorating the disease; for example, ameliorating a disease, condition or disorder in an individual that is experiencing or displaying the pathology or symptomatology of the disease, condition or disorder (i.e., reversing the pathology and/or symptomatology).

### Standard Biological And Pharmacological Test Procedure(S)

Evaluation of representative compounds of this invention in standard pharmacological test procedures indicated that the compounds of this invention possess significant anticancer activity and are in particular inhibitors of B-Raf kinase. Based on the activity shown in the standard pharmacological test procedures, the compounds of this invention are therefore useful as antineoplastic agents. In particular, these compounds are useful in treating, inhibiting the growth of, or eradicating neoplasms such as those of the breast, kidney, bladder, mouth, larynx, esophagus, stomach, colon, ovary, lung, pancreas, liver, prostate and skin.

Testing For B-Raf Kinase Inhibitors

Compounds of Formula A were tested as B-Raf Kinase inhibitors that can inhibit growth of tumor cells which contain oncogenic forms of Receptor Tyrosine Kinases or K-Ras, or B-Raf kinase.

Reagents: Flag/GST-tagged recombinant human B-Raf produced in Sf9 insect cells, human non-active Mek-1-GST (recombinant protein produced in E. coli); and a phospho-MEK1 specific poly-clonal Ab from Cell Signaling Technology (Cat. #9121).

B-Raf1 Kinase Assay Procedure: B-Raf-1 is used to phosphorylate GST-MEK1. MEK1 phosphorylation is measured by a phospho-specific antibody (from Cell Signaling Technology, Cat. #9121) that detects phosphorylation of two serine residues at positions 217 and 221 on MEK1.

The following Kinase Assay Protocol was employed in accordance with the invention:

### B-Raf Assay Stock Solutions:

1. Assay Dilution Buffer (ADB): 20 mM MOPS, pH 7.2, 25 mM B-glycerol phosphate, 5 mM EGTA, 1 mM sodium orthovanadate, 1 mM dithiothreitol, 0.01% Triton X-100.
2. Magnesium/ATP Cocktail: ADB solution (minus Triton X-100) plus 200 μM cold ATP and 40 mM magnesium chloride.
3. Active Kinase: Active B-Raf: used at 0.2 nM per assay point.
4. Non-active GST-MEK1: Use at 2.8 nM final concentration).
5. TBST-Tris (50 mM, pH 7.5), NaCl (150 mM), Tween-20 (0.05%)

### 6. Anti-GST Ab (GE)

7. Anti pMEK Ab (Upstate)
8. Anti-rabbit Ab/Europium conjugate (Wallac)

Assay Procedure:

1. Add 25 μl of ADB containing B-Raf and Mek per assay (i.e. per well of a 96 well plate)
2. Add 25 μl of 0.2 mM ATP and 40 mM magnesium chloride in Magnesuium/ATP Cocktail.
3. Incubate for 45 minutes at RT with occasional shaking.
4. Transfer this mixture to an anti-GST Ab coated 96 well plate (Nunc Immunosorb plates coated o/n with a-GST. Plate freshly washed 3× with TBS-T before use.
5. Incubate o/n at 30° C. in cold room.
6. Wash 3× with TBST, add Anti-Phospho MEK1 (1:1000, dilution depends upon lot)
7. Incubate for 60 minutes at RT in a shaking incubator
8. Wash 3× with TBST, add Anti-rabbit Ab/Europium conjugate (Wallac) (1:500, dilution depends upon lot)
9. Incubate for 60 minutes at RT on a platform shaker.
10. Wash plate 3× with TBS-T
11. Add 100 ul of Wallac Delfia Enhancement Solution and shake for 10 minutes.
12. Read plates in Wallac Victor model Plate Reader.
13. Collect data analyze in Excel for single point and IC50 determinations. Mallon R, et al (2001) Anal. Biochem. 294:48.

Analysis of Results:

IC50 determinations were performed on compounds of Formula A from single point assays with >80% inhibition. Single point assay—% inhibition at 10 mg/mL (% inhibition=1—sample treated with compound of Formula A/untreated control sample). The % inhibition was determined for each compound concentration. IC50 determinations—Typically the B-Raf assay was run at compound concentrations from 1 μM to 3 nM or 0.1 uM to 300 pm in half log dilutions. Data was analyzed in Excel and transferred to Radis.

IC50 Data for compounds of Formula A

| EXAMPLE | Mean IC50 (μM) |
|---|---|
| 2 | 0.018 |
| 3 | 1 |
| 5 | 0.091 |
| 6 | 0.002 |
| 7 | 0.002 |
| 9 | 0.055 |
| 10 | 0.329 |
| 14 | <0.0003 |
| 15 | <0.0003 |
| 18 | 0.0003 |

The compounds of this invention may be formulated neat or may be combined with one or more pharmaceutically acceptable carriers for administration. Suitable carriers include but are not limited to, for example, solvents, diluents and the like, and may be administered orally in such forms as tablets, capsules, dispersible powders, granules, or suspensions containing, for example, from about 0.05 to 5% of suspending agent, syrups containing, for example, from about 10 to 50% of sugar, and elixirs containing, for example, from about 20 to 50% ethanol, and the like, or parenterally in the form of sterile injectable solution or suspension containing from about 0.05 to 5% suspending agent in an isotonic medium. Such pharmaceutical preparations may contain, for example, from about 0.05 up to about 90% of the active ingredient in combination with the carrier, more usually between about 5% and 60% by weight.

In some embodiments, the formulations are administered transdermally which includes all methods of administration across the surface of the body and the inner linings of body passages including epithelial and mucosal tissues. Such administration may be in the form of a lotion, cream, colloid, foam, patch, suspension, or solution.

The effective dosage of active ingredient employed may vary depending on the particular compound employed, the mode of administration and the severity of the condition being treated. However, in general, satisfactory results are obtained when the compounds of the invention are administered at a daily dosage of from about 0.5 to about 1000 mg/kg of animal body weight, optionally given in divided doses two to four times a day, or in sustained release form. For most large mammals the total daily dosage is from about 1 to 1000 mg, preferably from about 2 to 500 mg. Dosage forms suitable for internal use comprise from about 0.5 to 1000 mg of the active compound in intimate admixture with a solid or liquid pharmaceutically acceptable carrier. This dosage regimen may be adjusted to provide the optimal therapeutic response. For example, several divided doses may be administered daily or the dose may be proportionally reduced as indicated by the exigencies of the therapeutic situation.

The compounds of this invention may be administered orally as well as by intravenous, intramuscular, or subcutaneous routes. Solid carriers include starch, lactose, dicalcium phosphate, microcrystalline cellulose, sucrose and kaolin, while liquid carriers include sterile water, polyethylene glycols, non-ionic surfactants and edible oils such as corn, peanut and sesame oils, as are appropriate to the nature of the active ingredient and the particular form of administration desired. Adjuvants customarily employed in the preparation of pharmaceutical compositions may be advantageously included, such as flavoring agents, coloring agents, preserving agents, and antioxidants, for example, vitamin E, ascorbic acid, BHT and BHA.

The preferred pharmaceutical compositions from the standpoint of ease of preparation and administration are solid compositions, particularly tablets and hard-filled or liquid-filled capsules. Oral administration of the compounds is sometimes desirable.

In some cases it may be desirable to administer the compounds directly to the airways in the form of an aerosol.

The compounds of this invention may also be administered parenterally or intraperitoneally. Solutions or suspensions of these active compounds as a free base or pharmacologically acceptable salt may be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier may be a solvent or dispersion medium containing, for example, water, ethanol, polyol (e.g., glycerol, propylene glycol and liquid polyethylene glycol), suitable mixtures thereof, and vegetable oils.

For the treatment of cancer, the compounds of this invention may be administered in combination with other antitumor substances or with radiation therapy. These other substances or radiation treatments may be given at the same or at different times as the compounds of this invention. These combined therapies may effect synergy and result in improved efficacy. For example, the compounds of this invention may be used in combination with mitotic inhibitors such as taxol or vinblastine, alkylating agents such as cisplatin or cyclophosamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, and antiestrogens such as tamoxifen.

As used in accordance with this invention, the term an “effective amount” of a compound means either directly administering such compound, or administering a prodrug, derivative, or analog which will form an effective amount of the compound within the body.

Methods of administration of a pharmaceutical composition of the invention are not specifically restricted, and can be administered in various preparations depending on the age, sex, and symptoms of the patient. For example, tablets, pills, solutions, suspensions, emulsions, granules and capsules may be orally administered. Injection preparations may be administered individually or mixed with injection transfusions such as glucose solutions and amino acid solutions intravenously. If necessary, the injection preparations are administered singly intramuscularly, intracutaneously, subcutaneously or intraperitoneally. Suppositories may be administered into the rectum.

The amount of the compound of formula A contained in a pharmaceutical composition according to the present invention is not specifically restricted, however, the dose should be sufficient to treat, ameliorate, or reduce the targeted symptoms. The dosage of a pharmaceutical composition according to the present invention will depend on the method of use, the age, sex, and condition of the patient.

The present invention also provides methods of inhibition and treatment further comprising administering an additional inhibitor of a oncopprotein kinase of the Ras/Raf/MEK pathway.

The pharmaceutical compositions of the present invention may comprise the compound of the present invention alone or in combination with other oncoprotein kinase-inhibiting compounds or chemotherapeutic agents. Chemotherapeutic agents include, but are not limited to exemestane, formestane, anastrozole, letrozole, fadrozole, taxane and derivatives such as paclitaxel or docetaxel, encapsulated taxanes, CPT-11, camptothecin derivatives, anthracycline glycosides, e.g., doxorubicin, idarubicin, epirubicin, etoposide, navelbine, vinblastine, carboplatin, cisplatin, estramustine, celecoxib, tamoxifen, raloxifen, Sugen SU-5416, Sugen SU-6668, and Herceptin.

Having described the invention, the invention is further illustrated by the following non-limiting examples.

### EXAMPLES

General procedure for the preparation of substituted aminopyrazole intermediates as exemplified for 4-[3-methoxy-phenyl]-5-pyridin-4-yl-1H-pyrazol-3-amine:

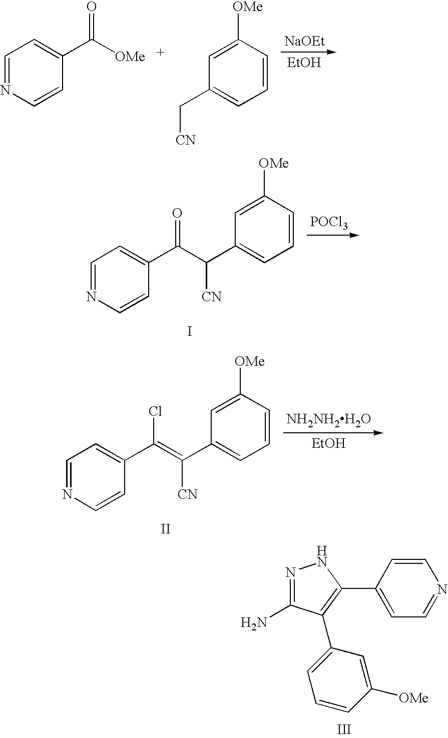

Step 1: To a 5 mL solution of dry EtOH was added 0.73 g (31.84 mmol) of Na metal (after removal of mineral oil with hexane). The mixture was stirred until the solution turned clear—needed heating at 45° C. for 1 hour. A mixture of 3 g (20.38 mmol) of the 3-(methoxyphenyl)acetonitrile and 3.9 g (28.66 mmol) of methyl isonicotinate in 26 mL of dry EtOH was added and the resulting brown solution was heated under reflux for 3 hours. After cooling, the residue was evaporated and purified by silica gel chromatography 9:1 to 4:1 methylene chloride/methanol to provide 1.75 g (34%) of 2-(3-methoxyphenyl)-3-oxo-3-pyridin-4-yl-propionitrile (I).

Step 2: A mixture of 1.7 g (6.74 mmol) of 2-(3-methoxyphenyl)-3-oxo-3-pyridin-4-yl-propionitrile and 17 mL POCl₃ was heated at 80° C. for 18 hours. After cooling, the POCl₃ was evaporated off. To the residue was added toluene, which was evaporated off to dryness. This step was repeated to fully remove POCl₃. Ice and saturated sodium bicarbonate was added to the residue, and a solid precipitated out, provided 1 g of 3-chloro-2-(3-methoxyphenyl)-3-pyridin-4-yl-acrylonitrile (II) as a white solid (57%). MS 271.1 [M+H].

Step 3: A mixture of 1 g (3.69 mmol) of 3-chloro-2-(3-methoxyphenyl)-3-pyridin-4-yl-acrylonitrile and 0.9 mL (18.6 mmol) hydrazine hydrate in 30 mL of ethanol was heated to reflux for 6.5 hours. The mixture was allowed to cool to room temperature and solvent was removed by evaporation. Aqueous sodium bicarbonate was stirred into the residue, and the resulting solid was collected by filtration. The solid was washed with water, then dried under vacuum to provide 0.92 g (94%) of 4-[3-methoxy-phenyl]-5-pyridin-4-yl-1H-pyrazol-3-amine (III). MS 267.2 [M+H].

Aminopyrazole intermediates 4-[3-(benzyloxy)phenyl]-5-pyridin-4-yl-1H-pyrazol-3-amine, 4-(4-fluoro-3-methoxyphenyl)-3-pyridin-4-yl-1H-pyrazol-5-amine and 4-(4-chloro-3-methoxyphenyl)-5-pyridin-4-yl-1H-pyrazol-3-amine were synthesized by the method of 4-[3-methoxy-phenyl]-5-pyridin-4-yl-1H-pyrazol-3-amine (III).

3-(3-amino-5-pyridin-4-yl-1H-pyrazol-4-yl)phenol. A mixture of 4-(3-methoxyphenyl)-5-pyridin-4-yl-1H-pyrazol-3-amine (3.0 g, 11.27 mmol) and pyridine hydrochloride (6.0 g, 51.92 mmol) was heated at 2020° C. for 1 hour. After cooling to room temperature, the mixture was stirred with ammonium hydroxide for 0.5 hr, and then filtered. The solid was set aside and the filtrate was evaporated to dryness to yield a solid residue. The combined solids were washed with 15% methanol in methylene chloride. Evaporation of the filtrate provided a crude residue that was purified by silica gel (12% methanol in methylene chloride) to yield 2.21 g (78%) of 3-(3-amino-5-pyridin-4-yl-1H-pyrazol-4-yl)phenol as a beige solid, mp, 1620-1640° C. MS: [M+H]⁺ 253.2.

5-(3-amino-5-pyridin-4-yl-1H-pyrazol-4-yl)-2-chlorophenol. 5-(3-Amino-5-pyridin-4-yl-1H-pyrazol-4-yl)-2-chlorophenol was prepared following the procedure of 3-(3-amino-5-pyridin-4-yl-1H-pyrazol-4-yl)phenol, by reacting 4-(4-chloro-3-methoxyphenyl)-5-pyridin-4-yl-1H-pyrazol-3-amine with pyridine hydrochloride. Purification by silica gel chromatography provided 5-(3-amino-5-pyridin-4-yl-1H-pyrazol-4-yl)-2-chlorophenol as a beige solid in 77% yield, mp, 273°-274° C. MS: [M+H] 287.1.

### Example 1

### Ethyl 8-[3-(benzyloxy)phenyl]-4-cyclohexyl-7-pyridin-4-yl-pyrazolo[5,1-c][1,2,4]triazine-3-carboxylate

To a cold (0°-50° C.) solution of 4-[3-(benzyloxy)phenyl]-5-pyridin-4-yl-1H-pyrazol-3-amine (0.5 g, 1.46 mmol) and ethanol (1.5 mL) in water (0.8 mL) was added cold solution of sodium nitrite (0.15 g, 2.18 mmol) in water (1.5 mL), stirred for 3 minutes and then was added cold solution of concentrated hydrochloric acid (0.39 mL) in water (0.28 mL). The resulting mixture was stirred for 3 minutes and was added a solution of 3-cyclohexyl-3-oxo-propionic acid ethyl ester (0.319 g, 1.61 mmol) in ethanol (0.3 mL), followed by the addition of sodium acetate (0.36 g, 4.38 mmol). The resulting thick mixture was stirred at (0°-5° C.) for 30 minutes and then at room temperature for 48 hours. A solid formed, from which the solvents were decanted, followed by drying under high vacuum. The solid was suspended in toluene (50 mL), and a catalytic amount of p-toluene sulfonic acid (0.02 g), the resulting mixture being heated under reflux (using a Dean Stark apparatus) for 2 hours. The reaction was allowed to cool and solvent was evaporated to dryness to yield a dark oil. The oil was purified by silica gel flash chromatography (1:99 to 2:98 methanol/methylene chloride) to yield 0.501 g (64%) of ethyl 8-[3-(benzyloxy)phenyl]-4-cyclohexyl-7-pyridin-4-yl-pyrazolo[5,1-c][1,2,4]triazine-3-carboxylate as an orange solid, MS 534.3 [M+H].

### Example 2

### 3-(4-cyclohexyl-7-pyridin-4-yl-pyrazolo[5,1-c][1,2,4]triazin-8-yl)phenol

### Step 1

### 8-(3-hydroxyphenyl)-4-cyclohexyl-7-pyridin-4-yl-pyrazolo[5,1-c][1,2,4]triazine-3-carboxylic acid

To a cold (0°-5° C.) solution of ethyl 8-[3-(benzyloxy)phenyl]-4-cyclohexyl-7-pyridin-4-yl-yrazolo[5,1-c][1,2,4]triazine-3-carboxylate (0.226 g, 0.42 mmol) in methylene chloride (6 mL) was added dropwise solution of boron tribromide (1 M solution in methylene chloride, 1.27 ml, 1.27 mmol) over a period of 10 minutes and the resulting mixture was stirred cold for 30 minutes. The mixture was quenched with ice cold water, and the resulting red solid was filtered, then dried in vacuo to yield 0.210 g of crude 8-(3-hydroxyphenyl)-4-cyclohexyl-7-pyridin-4-yl-pyrazolo[5,1-c][1,2,4]triazine-3-carboxylic acid which was in the next step without further purification.

### Step 2

### 3-(4-cyclohexyl-7-pyridin-4-yl-pyrazolo[5,1-c][1,2,4]triazin-8-yl)phenol

To a hot (200°-210° C.) solution of Dowtherm (1.5 mL) was added 8-(3-hydroxyphenyl)-4-cyclohexyl-7-pyridin-4-yl-pyrazolo[5,1-c][1,2,4]triazine-3-carboxylic acid (0.12 g, 0.29 mmol) and stirred for 10 minutes. The mixture was cooled to room temperature and the solvent was decanted off to provide a dark solid that was dried in vacuo. Purification by silica gel flash chromatography (1:99 to 2:98 methanol/methylene chloride) yielded 0.023 g (22%) of 3-(4-cyclohexyl-7-pyridin-4-yl-pyrazolo[5,1-c][1,2,4]triazin-8-yl)phenol as a yellow solid, MS 372.3 [M+H].

### Example 3

### [8-(3-Methoxyphenyl)-4-methyl-7-pyridin-4-yl-pyrazolo[5,1-c][1,2,4]triazin3-yl-(4-methylpiperazin-1-yl)-methanone

### Step 1

### tert-Butyl-8-[3-(methoxyphenyl]-4-methyl-7-pyridin-4-yl-pyrazolo[5,1-c][1,2,4]triazine-3-carboxylate

A solution of tert-butyl 3-oxobutanoate (0.42 mL, 2.1 mmol) in water (1.5 mL) and HCl (1.5 mL) was brought to 0° C. This solution was added to a suspension of 4-(3-methoxyphenyl)-3-(pyridin-4-yl)-1H-pyrazol-5-amine (516 mg, 1.9 mmol) and NaNO₂ (147 mg, 2.1 mmol) in water (2.5 mL) at 0° C. followed by the addition of sodium acetate (516 mg, 6.3 mmol). The reaction was stirred for 8 hr and the pH was adjusted to ˜7 with aqueous sodium bicarbonate. The resulting solid was filtered and washed with water followed by hexanes to give product (330 mg) in 42% yield. MS 418.3 [M+H].

### Step-2

### 8-(3-Methoxyphenyl)-4-methyl-7-pyridin-4-yl-pyrazolo[5,1-c][1,2,4]triazine-3-carboxylic acid

A mixture of tert-butyl 8-[3-(methoxyphenyl]-4-methyl-7-pyridin-4-yl-pyrazolo[5,1-c][1,2,4]triazine-3-carboxylate (1.0 g, 2.39 mmol) and formic acid (18 mL) was stirred at room temperature over 48 hours. The solvent was evaporated to dryness and the residue was reevaporated twice from toluene. The solid was triturated with ether, collected by filtration, washed with methylene chloride and dried to yield 8-(3-methoxyphenyl)-4-methyl-7-pyridin-4-yl-pyrazolo[5,1-c][1,2,4]triazine-3-carboxylic acid as a yellow solid, 160°-170° C.; MS 362.2 [M+H].

### Step-3

### [8-(3-Methoxyphenyl)-4-methyl-7-pyridin-4-yl-pyrazolo[5,1-c][1,2,4]triazin3-yl-(4-methylpiperazin-1-yl)-methanone

A mixture of 8-(3-Methoxyphenyl)-4-methyl-7-pyridin-4-yl-pyrazolo[5,1-c][1,2,4]triazine-3-carboxylic acid (0.15 g, 0.42 mmol), triethylamine (0.25 mL, 1.79 mmol), 1-methyl piperazine (0.2 mL, 1.8 mmol) and diethylcyanophosphonate (0.2 ml, 1.32 mmol) in dimethyl formamide (1.0 mL) was stirred at room temperature over night. The solvent was evaporated using high vacuum, the residue was dissolved in water and extracted with 10% methanol in methylene chloride. The organic extract was dried over anhydrous sodium sulfate, filtered and filtrate was evaporated to yield an oil. The crude oil was purified by silica gel flash chromatography (methanol in methylene chloride) to yield 20 mg (11%) of [8-(3-methoxyphenyl)-4-methyl-7-pyridin-4-yl-pyrazolo[5,1-c][1,2,4]triazin3-yl-(4-methylpiperazin-1-yl)-methanone as a yellow solid, MS 444.4 [M+H].

### Example 4

### 4-(6-Bromo-pyridin-3yl-)-8-(3-methoxy-phenyl)-7-pyridin-4-yl-pyrazolo[5,1-c][1,2,4]triazine-3-carboxylic acid ethyl ester

The compound 4-(6-Bromo-pyridin-3-yl-)-8-(3-methoxy-phenyl)-7-pyridin-4-yl-pyrazolo[5,1-c][1,2,4]triazine-3-carboxylic acid ethyl ester was prepared by the method of Example 1, step 1, by the reaction of 4-[3-methoxy-phenyl]-5-pyridin-4-yl-1H-pyrazol-3-amine (1.0 g, 3.75 mmol) with ethyl-3-(6-bromo-pyridine-3-yl)-3-oxo propionate (1.06 g, 3.91 mmol). The crude foam was purified by silica gel flash chromatography (2% methanol in methylene chloride) to yield 1.13 g (57%) of 4-(6-bromo-pyridin-3-yl-)-8-(3-methoxy-phenyl)-7-pyridin-4-yl-pyrazolo[5,1-c][1,2,4]triazine-3-carboxylic acid ethyl ester as an orange solid, 860-88° C.; MS 531.2,[M+H], 533.2 [M+H].

### Example 5

### 3-[4-(6-Bromo-pyridin-3-yl)-7-pyridin-4-yl-pyrazolo[5,1-c][1,2,4]triazin-8-yl]-phenol

### Step 1

### 4-(6-Bromo-pyridin-3-yl-)-8-(3-hydroxy-phenyl)-7-pyridin-4-yl-pyrazolo[5,1-c][1,2,4]triazine-3-carboxylic acid

The compound 4-(6-bromo-pyridin-3yl-)-8-(3-hydroxy-phenyl)-7-pyridin-4-yl-pyrazolo[5,1-c][1,2,4]triazine-3-carboxylic acid was prepared following the method for Example 1, step 2, using 4-(6-bromo-pyridin-3yl-)-8-(3-methoxy-phenyl)-7-pyridin-4-yl-pyrazolo[5,1-c][1,2,4]triazine-3-carboxylic acid ethyl ester (0.8 g, 1.5 mmol) and boron tri bromide (1 M solution in methylene chloride, 9.0 ml, 9.0 mmol). The crude product was used in the next step without further purification.

### Step 2

### 3-[4-(6-Bromo-pyridin-3-yl)-7-pyridin-4-yl-pyrazolo[5,1-c][1,2,4]triazin-8-yl]-phenol

The compound 3-[4-(6-bromo-pyridin-3-yl)-7-pyridin-4-yl-pyrazolo[5,1-c][1,2,4]triazin-8-yl]-phenol was prepared following the above method for Example 2, step 2 using crude 4-(6-bromo-pyridin-3yl-)-8-(3-hydroxy-phenyl)-7-pyridin-4-yl-pyrazolo[5,1-c][1,2,4]triazine-3-carboxylic acid (0.87 g) in Dowtherm (25.0 mL) at 1650° C. for 3 minutes. The crude product was purified by silica gel flash chromatography (3% methanol in methylene chloride) to yield 0.12 g of 3-[4-(6-bromo-pyridin-3-yl)-7-pyridin-4-yl-pyrazolo[5,1-c][1,2,4]triazin-8-yl]-phenol as an orange solid. MS 445.1,[M+H], 447.1 [M+H].

### Example 6

### 3-{4-[6-(4-Methyl-[1,4]diazepan-1-yl)-pyridin-3-yl]-7-pyridin-4-yl-pyrazolo[5,1-c][1,2,4]triazin-8yl}-phenol

A mixture of 3-[4-(6-bromo-pyridin-3-yl)-7-pyridin-4-yl-pyrazolo[5,1-c][1,2,4]triazin-8-yl]-phenol (0.04 g, 0.09 mmol), 1-methylhomopiperazine (0.04 mL, 0.32 mmol) and N,N-diisopropylethylamine (0.1 mL, 0.57 mmol) in 1-methyl-2-pyrrolidinone (0.5 mL) was heated at 100° C. for 18 hours. The resulting product mixture was cooled to room temperature and diluted with saturated solution of sodium bicarbonate. A solid was obtained, that was collected by filtration, washed with water and dried. The crude product was purified by silica gel flash chromatography (gradient 95:5 to 4:1 methylene chloride/methanol) to yield 0.4 g of 3-{4-[6-(4-methyl-[1,4]diazepan-1-yl)-pyridin-3-yl]-7-pyridin-4-yl-pyrazolo[5,1-c][1,2,4]triazin-8-yl}-phenol as an orange solid, MS 479.3 [M+H].

### Example 7

### 3-{4-[6-(4-Methyl-piperazin-1-yl)-pyridin-3-yl]-7-pyridin-4-yl-pyrazolo[5,1-c][1,2,4]triazin-8-yl}-phenol

The compound 3-{4-[6-(4-Methyl-piperazin-1-yl)-pyridin-3-yl]-7-pyridin-4-yl-pyrazolo[5,1-c][1,2,4]triazin-8-yl}-phenol was prepared following the method for example 6, by the reaction of 3-[4-(6-bromo-pyridin-3-yl)-7-pyridin-4-yl-pyrazolo[5,1-c][1,2,4]triazin-8-yl]-phenol with 1-methypiperazine. The crude product was purified by silica gel flash chromatography (gradient 95:5 to 4:1 methylene chloride/methanol) to yield 3-{4-[6-(4-methyl-piperazin-1-yl)-pyridin-3-yl]-7-pyridin-4-yl-pyrazolo[5,1-c][1,2,4]triazin-8-yl}-phenol as an orange solid, MS 465.3 [M+H].

### Example 8

### 8-(3-methoxyphenyl)-4-methyl-3-(phenylsulfonyl)-7-pyridin-4-yl pyrazolo[5,1-c][1,2,4]triazine

The compound 8-(3-Methoxyphenyl)-4-methyl-3-(phenylsulfonyl)-7-pyridin-4-ylpyrazolo[5,1-c][1,2,4]triazine was prepared by the method of example 1 from 4-(3-methoxyphenyl)-3-(pyridin-4-yl)-1H-pyrazol-5-amine (500 mg, 1.9 mmol) and phenylsulfonylacetone (0.041 g, 0.21 mmol). The crude product was purified by silica gel flash chromatography (eluting with 97:3 methylene chloride/methanol) to yield 0.053 g (61%) of 8-(3-methoxyphenyl)-4-methyl-3-(phenylsulfonyl)-7-pyridin-4-ylpyrazolo[5,1-c][1,2,4]triazine as a yellow solid, MS 458.3 [M+H].

### Example 9

### 3-[4-Methyl-3-(phenylsulfonyl)-7-pyridin-4-ylpyrazolo[5,1-c][1,2,4]triazin-8-yl]phenol

The compound 3-[4-Methyl-3-(phenylsulfonyl)-7-pyridin-4-ylpyrazolo[5,1-c][1,2,4]triazin-8-yl]phenol was prepared by the method of Example 2, step 1 by the reaction of 8-(3-methoxyphenyl)-4-methyl-3-(phenylsulfonyl)-7-pyridin-4-ylpyrazolo[5,1-c][1,2,4]triazine (0.033 g, 0.072 mmol) with boron tribromide (1 M solution in methylene chloride, 0.35 mL, 0.35 mmol). The crude product was purified by silica gel flash chromatography (eluting with 95:5 methylene chloride/methanol) to yield 0.021 g (66%) of 3-[4-methyl-3-(phenylsulfonyl)-7-pyridin-4-ylpyrazolo[5,1-c][1,2,4]triazin-8-yl]phenol as a yellow solid, MS 444.3 [M+H].

### Example 10

### Ethyl 8-(4-chloro-3-methoxyphenyl)-4-cyclopropyl-7-pyridin-4-yl pyrazolo[5,1-c][1,2,4]triazine-3-carboxylate

Example 10 was prepared following the procedure described for Example 1 by the reaction of 4-(4-chloro-3-methoxyphenyl)-5-pyridin-4-yl-1H-pyrazol-3-amine with 3-cyclopropyl-3-oxo-propionic acid ethyl ester. MS (electrospray): m/z 450.2 [M+H].

### Example 11

### Ethyl 4-(6-bromopyridin-3-yl)-8-(4-chloro-3-methoxyphenyl)-7-pyridin-4-yl pyrazolo[5,1-c][1,2,4]triazine-3-carboxylate

Example 11 was prepared following the procedure described for example 1 by the reaction of 4-(4-chloro-3-methoxyphenyl)-5-pyridin-4-yl-1H-pyrazol-3-amine with ethyl-3-(6-bromo-pyridine-3-yl)-3-oxo propionate. MS (electrospray): m/z 565.2 [M+H].

### Example 12

### 8-(4-Chloro-3-methoxy-phenyl)-4-[3-(4-methyl-piperazine-1-sulfonyl)-phenyl]-7-pyridin-4-yl-pyrazolo[5,1-c][1,2,4]triazine-3-carboxylic acid ethyl ester

### Step 1

### 3-[3-(4-Methyl-piperazine-1-sulfonyl)-phenyl]-3-oxo-propionic acid ethyl ester

To a hot (70° C.) mixture of diethyl carbonate (0.18 ml, 1.4 mmol) and sodium hydride (0.057 g, 1.4 mmol) in tetrahydrofuran (3 mL) was added 1-[3-(4-methyl-piperazine-1-sulfonyl)-phenyl]-ethanone (0.2 g, 0.7 mmol) (synthesized by the method of reference example 15, U.S. Pat. No. 5,459,131) in portions over a period of 1 hour. The resulting brown mixture was heated at 70° C. for 2 hours. After cooling, the mixture was poured in to ice water, neutralized with few drops of acetic acid, and extracted with ethyl acetate. The ethyl acetate extract was dried with anhydrous sodium sulfate, filtered and filtrate was evaporated to yield an oil. Purification of the oil by silica gel flash chromatography (5% methanol in methylene chloride) yielded 0.11 g (44%) of 3-[3-(4-methyl-piperazine-1-sulfonyl)-phenyl]-3-oxo-propionic acid ethyl ester. MS 355.3 [M+H].

### Step 2

### 8-(4-Chloro-3-methoxy-phenyl)-4-[3-(4-methyl-piperazine-1-sulfonyl)-phenyl]-7-pyridin-4-yl-pyrazolo[5,1-c][1,2,4]triazine-3-carboxylic acid ethyl ester.

The acid ethyl ester was prepared following the procedure described for example 1 by the reaction of 4-(4-chloro-3-methoxyphenyl)-5-pyridin-4-yl-1H-pyrazol-3-amine with 3-[3-(4-methyl-piperazine-1-sulfonyl)-phenyl]-3-oxo-propionic acid ethyl ester. MS 648.3 [M+H].

### Example 13

### 5-[4-(6-Bromopyridin-3-yl)-7-pyridin-4-ylpyrazolo[5,1-c][1,2,4]triazin-8-yl]-2-chlorophenol

Example 13 was prepared following the procedure described for example 5, steps 1 and 2 using ethyl 4-(6-bromopyridin-3-yl)-8-(4-chloro-3-methoxyphenyl)-7-pyridin-4-ylpyrazolo[5,1-c][1,2,4]triazine-3-carboxylate and the corresponding reagents. MS (electrospray): m/z 479.1 [M+H].

### Example 14

### 2-Chloro-5-{4-[6-(4-methyl-1,4-diazepan-1-yl)pyridin-3-yl]-7-pyridin-4-yl pyrazolo[5,1-c][1,2,4]triazin-8-yl}phenol

Example 14 is prepared following the procedure described for example 6 by the reaction of 5-[4-(6-bromopyridin-3-yl)-7-pyridin-4-ylpyrazolo[5,1-c][1,2,4]triazin-8-yl]-2-chlorophenol with 1-methylhomopiperazine. MS (electrospray): m/z 513.4 [M+H].

### Example 15

### 2-Chloro-5-(4-{3-[(4-methylpiperazin-1-yl)sulfonyl]phenyl}-7-pyridin-4-ylpyrazolo[5,1-c][1,2,4]triazin-8-yl)phenol

Example 15 is prepared following the procedure described for example 5, steps 1 and 2 using 8-(4-chloro-3-methoxy-phenyl)-4-[3-(4-methyl-piperazine-1-sulfonyl)-phenyl]-7-pyridin-4-yl-pyrazolo[5,1-c][1,2,4]triazine-3-carboxylic acid ethyl ester and the corresponding reagents. MS (electrospray): m/z 562.3 [M+H].

### Example 16

### Ethyl 3-[8-(4-chloro-3-methoxyphenyl)-3-(phenylsulfonyl)-7-pyridin-4-ylpyrazolo[5,1-c][1,2,4]triazin-4-yl]-8-azabicyclo[3.2.1]octane-8-carboxylate

### Step 1

### ethyl 3-cyano-8-azabicyclo[3.2.1]octane-8-carboxylate

A mixture of tosylmethylisocyanide (5 g, 25.6 mmol) and ethyl 3-oxo-8-azabicyclo[3.2.1]octane-8-carboxylate (3.8 g, 19.7 mmol) in dimethoxyethane (60 mL) and ethanol (1.85 mL) was stirred at −10° C. while adding t-BuOK portionwise over the course of 1 hour so that the temperature was maintained at <50° C. Once the addition was complete, the reaction was stirred at −10° C. for 1 h and then stirred for additional 2 h at room temperature. The solvents were then removed under reduced pressure to give an orange brown solid. To this solid was added water (200 mL) and the resulting aqueous mixture was extracted with ether (4×, 150 mL). The organic extract was dried over anhydrous magnesium sulfate, filtered and the filtrate was evaporated to yield a brown oil. The crude mixture was purified on a silica column using 30% ethyl acetate in hexanes to give ethyl 3-cyano-8-azabicyclo[3.2.1]octane-8-carboxylate (2.43 g, 60% yield). MS 209.2 [M+H].

### Step 2

### 3-(2-Benzenesulfonyl-acetyl)-8-aza-bicyclo[3.2,1]octane-8-carboxylic acid ethyl ester

A solution of methyl phenyl sulfone (0.35 g, 2.22 mmol) in anhydrous tetrahyrofuran (7 mL) was cooled at 0° C. under nitrogen and thereafter treated dropwise with a solution of n-butyllithium(1.6 M) in hexane (3.0 mL, 4.8 mmol).Upon the addition, a yellow precipitate began to form. After a further hour of stirring at 0° C., the mixture was treated with solution of ethyl 3-cyano-8-azabicyclo[3.2.1]octane-8-carboxylate (0.5 g, 2.4 mmol) in tetrahyrofuran. The reaction mixture was stirred for 30 minutes at 0° C. and then allowed to attain room temperature. After quenching the solution with water, it was extracted with ether. The ether extract was dried over anhydrous sodium sulfate, evaporated in vacuo to yield an oil. This oil was stirred with 10 ml of 1 N hydrochloric acid and dioxane for 1 hour. Evaporation of the solvent once again provided an oil, which was purified by silica gel flash chromatography (1% methanol in methylene chloride) to yield 0.14 g (17%) of. 3-(2-benzenesulfonyl-acetyl)-8-aza-bicyclo[3.2,1]octane-8-carboxylic acid ethyl ester. MS 366.3 [M+H].

### Step 3

### Ethyl 3-[8-(4-chloro-3-methoxyphenyl)-3-(phenylsulfonyl)-7-pyridin-4-yl pyrazolo[5,1-c][1,2,4]triazin-4-yl]-8-azabicyclo[3.2.1]octane-8-carboxylate

The carboxylate was prepared by the method of example 1 by the reaction of 3-(2-benzenesulfonyl-acetyl)-8-aza-bicyclo[3.2,1]octane-8-carboxylic acid ethyl ester with 4-(4-chloro-3-methoxyphenyl)-5-pyridin-4-yl-1H-pyrazol-3-amine and the corresponding reagents. MS 659.4 [M+H].

### Example 17

### Ethyl 8-(4-chloro-3-methoxyphenyl)-4-[8-(ethoxycarbonyl)-8-azabicyclo[3.2.1]oct-3-yl]-7-pyridin-4-yl pyrazolo[5,1-c][1,2,4]triazine-3-carboxylate

### Step 1

### Ethyl 3-acetyl-8-azabicyclo[3.2.1]octane-8-carboxylate

A 1.4M solution of methyl magnesium bromide (35.4 mL) THF/toluene was added to a solution of ethyl 3-cyano-8-azabicyclo[3.2.1]octane-8-carboxylate (2.4 g, 11.5 mmol) in tetrahydrofuran (50 mL) at room temperature. The reaction was stirred for 3 hours and quenched with ammonium chloride (100 mL). The mixture was then extracted with ether (4×, 100 mL). The organic extract was dried over anhydrous magnesium sulfate, filtered and filtrate was evaporated to yield ethyl 3-acetyl-8-azabicyclo[3.2.1]octane-8-carboxylate as an oil, MS 226.2 [M+H].

### Step 2

### Ethyl 3-(3-ethoxy-3-oxopropanoyl)-8-azabicyclo[3.2.1]octane-8-carboxylate

Ethyl 3-(3-ethoxy-3-oxopropanoyl)-8-azabicyclo[3.2.1]octane-8-carboxylate was prepared by the method of example 12, step 1, by the reaction of ethyl 3-acetyl-8-azabicyclo[3.2.1]octane-8-carboxylate with the corresponding reagents. MS 298.3 [M+H].

### Step 3

### Ethyl 8-(4-chloro-3-methoxyphenyl)-4-[8-(ethoxycarbonyl)-8-azabicyclo[3.2.1]oct-3-yl]-7-pyridin-4-yl pyrazolo[5,1-c][1,2,4]triazine-3-carboxylate

The carboxylate was prepared by the method of example 1 by the reaction of ethyl 3-(3-ethoxy-3-oxopropanoyl)-8-azabicyclo[3.2.1]octane-8-carboxylate with 4-(4-chloro-3-methoxyphenyl)-5-pyridin-4-yl-1H-pyrazol-3-amine and the corresponding reagents. MS 591.4 [M+H].

### Example 18

### Ethyl 3-[8-(4-chloro-3-hydroxyphenyl)-7-pyridin-4-ylpyrazolo[5,1-c][1,2,4]triazin-4-yl]-8-azabicyclo[3.2.1]octane-8-carboxylate

Example 18 was prepared following the procedure described for example 5, steps 1 and 2 using ethyl 8-(4-chloro-3-methoxyphenyl)-4-[8-(ethoxycarbonyl)-8-azabicyclo[3.2.1]oct-3-yl]-7-pyridin-4-ylpyrazolo[5,1-c][1,2,4]triazine-3-carboxylate and the corresponding reagents. MS 505.3 [M+H].

### Example 19

### 2-Chloro-5-[4-(2-fluoro-4-methoxyphenyl)-3-(phenylsulfonyl)-7-pyridin-4-ylpyrazolo[5,1-c][1,2,4]triazin-8-yl]phenol

### Step 1

### 1-(2-Fluoro-4-methoxyphenyl)-2-(phenylsulfonyl)ethanone

To a solution of 1-(2-fluoro-4-methoxyphenyl)ethanone (0.5 g, 2.97 mmol) in 20 mL of acetonitrile was added [hydroxy(tosyloxy)iodo]benzene (1.166 g, 2.97 mmol), and the resulting mixture was brought to reflux for 40 minutes. After allowing to cool to room temperature, sodium phenylsulfinate (0.976 g, 5.95 mmol) was added, followed by dropwise addition of 4 mL of water. The mixture was further refluxed for 2 hours, then partitioned between 75 mL of methylene chloride and 30 mL of water. The methylene chloride layer was concentrated in vacuo, and the crude product mixture purified by silica gel chromatography (eluting with 3:1 hexane/ethyl acetate) to provide 0.328 g of 1-(2-fluoro-4-methoxyphenyl)-2-(phenylsulfonyl)ethanone as a white solid. MS 309.1 [M+H].

### Step 2

### 2-Chloro-5-[4-(2-fluoro-4-methoxyphenyl)-3-(phenylsulfonyl)-7-pyridin-4-ylpyrazolo[5,1-c][1,2,4]triazin-8-yl]phenol

The compound 2-chloro-5-[4-(2-fluoro-4-methoxyphenyl)-3-(phenylsulfonyl)-7-pyridin-4-ylpyrazolo[5,1-c][1,2,4]triazin-8-yl]phenol was prepared by the method of example 1 from 5-(3-amino-5-pyridin-4-yl-1H-pyrazol-4-yl)-2-chlorophenol (80 mg, 0.28 mmol),1-(2-fluoro-4-methoxyphenyl)-2-(phenylsulfonyl)ethanone (94 mg, 0.30 mmol) and the corresponding reagents. The crude product was purified by silica gel flash chromatography (eluting with 98:2 methylene chloride/methanol) to yield 16 mg (10%) of 2-chloro-5-[4-(2-fluoro-4-methoxyphenyl)-3-(phenylsulfonyl)-7-pyridin-4-ylpyrazolo[5,1-c][1,2,4]triazin-8-yl]phenol as an orange solid, MS 588.3 [M+H].

### Example 20

### 8-(4-Chloro-3-methoxyphenyl)-4-(2-fluoro-4-methoxyphenyl)-3-(phenylsulfonyl)-7-pyridin-4-yl pyrazolo[5,1-c][1,2,4]triazine

The compound 8-(4-chloro-3-methoxyphenyl)-4-(2-fluoro-4-methoxyphenyl)-3-(phenylsulfonyl)-7-pyridin-4-ylpyrazolo[5,1-c][1,2,4]triazine was prepared by the method of example 1 from 4-(4-chloro-3-methoxyphenyl)-5-pyridin-4-yl-1H-pyrazol-3-amine (150 mg, 0.50 mmol), 1-(2-fluoro-4-methoxyphenyl)-2-(phenylsulfonyl)ethanone (169 mg, 0.55 mmol) and the corresponding reagents. The crude product was purified by silica gel flash chromatography (eluting with 97:3 methylene chloride/methanol) to yield 124 mg (41%) of 8-(4-chloro-3-methoxyphenyl)-4-(2-fluoro-4-methoxyphenyl)-3-(phenylsulfonyl)-7-pyridin-4-ylpyrazolo[5,1-c][1,2,4]triazine as a yellow solid, MS 602.3 [M+H].

### Example 21

### 2-CHLORO-5-(4-CYCLOPROPYL-7-PYRIDIN-4-YL-PYRAZOLO[5,1-C][1,2,4]TRIAZINE-8-YL)PHENOL

### Step 1

### ETHYL 8-(4-CHLORO-3-METHOXYPHENYL)-4-CYCLOPROPYL-7-PYRIDIN-4-YL-PYRAZOLO[5,1-C][1,2,4]TRIAZINE-3-CARBOXYLATE

The compound ethyl 8-(4-chloro-3-methoxyphenyl)-4-cyclopropyl-7-pyridin-4-yl-pyrazolo[5,1-c][1,2,4]triazine-3-carboxylate was prepared by the method of example 1, by the reaction of 4-(4-chloro-3-methoxyphenyl)-5-pyridin-4-yl-1H-pyrazol-3-amine (0.200 g, 0.66 mmol) with ethyl-3-cyclopropyl-3-oxo propionate (0.128 g, 0.82 mmol). The crude foam was purified by silica gel flash chromatography (5% methanol in methylene chloride) to yield 0.174 g (58%) of ethyl 8-(4-chloro-3-methoxyphenyl)-4-cyclopropyl-7-pyridin-4-yl-pyrazolo[5,1-c][1,2,4]triazine-3-carboxylate as an orange-beige solid; MS 450.0 [M+H].

### Step 2

### 8-(4-CHLORO-3-HYDROXYPHENYL)-4-CYCLOPROPYL-7-PYRIDIN-4-YL-PYRAZOLO[5,1-C][1,2,4]TRIAZINE-3-CARBOXYLIC ACID

The compound 8-(4-chloro-3-hydroxyphenyl)-4-cyclopropyl-7-pyridin-4-yl-pyrazolo[5,1-c][1,2,4]triazine-3-carboxylic acid was prepared following the method for example 2, step 1, using 8-(4-chloro-3-methoxyphenyl)-4-cyclopropyl-7-pyridin-4-yl-pyrazolo[5,1-c][1,2,4]triazine-3-carboxylic acid ethyl ester (0.17 g, 0.38 mmol) and boron tribromide (1 M solution in methylene chloride, 4.6 ml, 4.6 mmol). The crude product was used in the next step without further purification.

### Step 3

### 2-CHLORO-5-(4-CYCLOPROPYL-7-PYRIDIN-4-YL-PYRAZOLO[5,1-C][1,2,4]TRIAZINE-8-YL)PHENOL

To a hot (165° C.) solution of Dowtherm (3 mL) was added 8-(4-chloro-3-hydroxyphenyl)-4-cyclopropyl-7-pyridin-4-yl-pyrazolo[5,1-c][1,2,4]triazine-3-carboxylic acid (0.19 g, 0.38 mmol) and stirred for 10 minutes. The mixture was cooled to room temperature and diluted with hexanes to precipitate dark solid. The solid was filtered, re-dissolved in DMSO and purified by HPLC to yield 0.010 g (2.7%) of 2-chloro-5-(4-cyclopropyl-7-pyridin-4-yl-pyrazolo[5,1-c][1,2,4]triazin-8-yl)phenol as a beige solid, MS 364.2 [M+H].

### Example 22

### Ethyl 8-(4-chloro-3-methoxyphenyl)-4-piperidin-4-yl-7-pyridin-4-yl pyrazolo[5,1-c][1,2,4]triazine-3-carboxylate

### Step 1

### tert-Butyl 4-(3-ethoxy-3-oxopropanoyl)piperidine-1-carboxylate

tert-Butyl 4-(3-ethoxy-3-oxopropanoyl)piperidine-1-carboxylate was prepared following the procedure described for example 12, step 1 by the reaction of tert-butyl 4-acetylpiperidine-1-carboxylate (2.15 g, 9.46 mmol), with diethyl carbonate (2.3 mL, 19.0 mmol). The crude product was purified by silica gel flash chromatography, eluting with a gradient of 99.3:0.7-99:1 methylene chloride/methanol) to yield 1.16 g (41%) of tert-butyl 4-(3-ethoxy-3-oxopropanoyl)piperidine-1-carboxylate as a colorless oil. MS 300.3 [M+H].

### Step 2

### Ethyl 8-(4-chloro-3-methoxyphenyl)-4-piperidin-4-yl-7-pyridin-4-ylpyrazolo[5,1-c][1,2,4]triazine-3-carboxylate

The compound ethyl 8-(4-chloro-3-methoxyphenyl)-4-piperidin-4-yl-7-pyridin-4-ylpyrazolo[5,1-c][1,2,4]triazine-3-carboxylate was prepared by the method of example 1 from 4-(4-chloro-3-methoxyphenyl)-5-pyridine 4-yl-1H-pyrazol-3-amine (0.906 g, 3.0 mmol), tert-butyl 4-(3-ethoxy-3-oxopropanoyl)piperidine-1-carboxylate (0.9 g, 3.0 mmol) and the corresponding reagents. The crude product was purified by silica gel flash chromatography eluting with a gradient of 95:5-85:15 methylene chloride/methanol) to yield 0.722 g (52%) of ethyl 8-(4-chloro-3-methoxyphenyl)-4-piperidin-4-yl-7-pyridin-4-ylpyrazolo[5,1-c][1,2,4]triazine-3-carboxylate as a yellow solid, MS 493.4 [M+H].

### Example 23

### Ethyl 8-(4-chloro-3-methoxyphenyl)-4-(1-ethyl piperidin-4-yl)-7-pyridin-4-yl pyrazolo[5,1-c][1,2,4]triazine-3-carboxylate

A mixture of ethyl 8-(4-chloro-3-methoxyphenyl)-4-piperidin-4-yl-7-pyridin-4-ylpyrazolo[5,1-c][1,2,4]triazine-3-carboxylate (0.24 g, 0.49 mmol), iodoethane (0.08 mL, 1.0 mmol) and anhydrous potassium carbonate in 2 mL of N,N-dimethyl formamide was stirred at room temperature for 30 minutes. The mixture was poured in to water and solid was collected by filtration, washed with water and dried. The crude product was purified by silica gel flash chromatography eluting with a gradient of 97:3-92:8 methylene chloride/methanol) to yield 0.155 g (60%) of ethyl 8-(4-chloro-3-methoxyphenyl)-4-(1-ethylpiperidin-4-yl)-7-pyridin-4-ylpyrazolo[5,1-c][1,2,4]triazine-3-carboxylate as a yellow solid, MS 521.4 [M+H].

### Example 24

### 2-Chloro-5-[4-(1-ethyl piperidin-4-yl)-7-pyridin-4-ylpyrazolo[5,1-c][1,2,4]triazin-8-yl]phenol

### Step 1

### 8-(4-chloro-3-hydroxyphenyl)-4-(1-ethyl piperidin-4-yl)-7-(pyridin-4-yl)pyrazolo[5,1-c][1,2,4]triazine-3-carboxylic acid

The compound 8-(4-chloro-3-hydroxyphenyl)-4-(1-ethylpiperidin-4-yl)-7-(pyridin-4-yl)pyrazolo[5,1-c][1,2,4]triazine-3-carboxylic acid was prepared following the method for example 2, step 1, using ethyl 8-(4-chloro-3-methoxyphenyl)-4-(1-ethylpiperidin-4-yl)-7-pyridin-4-ylpyrazolo[5,1-c][1,2,4]triazine-3-carboxylate (0.115 g, 0.22 mmol) and boron tribromide (1 M solution in methylene chloride, 2.5 ml, 2.5 mmol). The crude product was used in the next step without further purification.

### Step 2

### 2-chloro-5-[4-(1-ethyl piperidin-4-yl)-7-pyridin-4-yl pyrazolo[5,1-c][1,2,4]triazin-8-yl]phenol

The compound 2-chloro-5-[4-(1-ethylpiperidin-4-yl)-7-pyridin-4-ylpyrazolo[5,1-c][1,2,4]triazin-8-yl]phenol was prepared following the above method for example 5, step 2 using crude 8-(4-chloro-3-hydroxyphenyl)-4-(1-ethylpiperidin-4-yl)-7-(pyridin-4-yl)pyrazolo[5,1-c][1,2,4]triazine-3-carboxylic acid (0.13 g) in Dowtherm (2.5 mL) at 1650° C. for 3 minutes. The crude product was purified by silica gel flash chromatography, eluting with a gradient of 94:6-80:20 methylene chloride/methanol) to yield 97 mg (86% for 2 steps) of 2-chloro-5-[4-(1-ethylpiperidin-4-yl)-7-pyridin-4-ylpyrazolo[5,1-c][1,2,4]triazin-8-yl]phenol as a yellow solid. MS 435.3 [M+H].

### Example 25

### Ethyl 8-(4-chloro-3-methoxyphenyl)-4-[4-(4-methyl piperazin-1-yl)phenyl]-7-pyridin-4-yl pyrazolo[5,1-c][1,2,4]triazine-3-carboxylate

### Step 1

### Ethyl 3-[4-(4-methyl piperazin-1-yl)phenyl]-3-oxopropanoate

To a hot (70° C.) mixture of diethyl carbonate (2.2 ml, 18.3 mmol) and 60% sodium hydride in mineral oil (0.733 g, 18.3 mmol) in tetrahydrofuran (24 mL) was added 4-(4-methylpiperazino)acetophenone (2 g, 9.16 mmol) in 10 mL of tetrahydrofuran over a period of 1 hour. The resulting brown mixture was heated at 70° C. for 2 hours. After cooling, the mixture was quenched with methanol dropwise, then 30 mL of water. The mixture was neutralized with acetic acid, then brought to pH 8 with sodium bicarbonate and extracted 5× with 30 mL ethyl acetate. The ethyl acetate extract was dried with anhydrous sodium sulfate, filtered and filtrate was evaporated to yield an oil. Purification of the oil by silica gel flash chromatography (gradient 2-5% methanol in methylene chloride) yielded 2.5 g (94%) of ethyl 3-[4-(4-methylpiperazin-1-yl)phenyl]-3-oxopropanoate. MS 291.2 [M+H].

### Step 2

### Ethyl 8-(4-chloro-3-methoxyphenyl)-4-[4-(4-methyl piperazin-1-yl)phenyl]-7-pyridin-4-yl pyrazolo[5,1-c][1,2,4]triazine-3-carboxylate

The compound ethyl 8-(4-chloro-3-methoxyphenyl)-4-[4-(4-methylpiperazin-1-yl)phenyl]-7-pyridin-4-ylpyrazolo[5,1-c][1,2,4]triazine-3-carboxylate prepared by the method of example 1 from 4-(4-chloro-3-methoxyphenyl)-5-pyridin-4-yl-1H-pyrazol-3-amine (300 mg, 1.05 mmol), ethyl 3-[4-(4-methylpiperazin-1-yl)phenyl]-3-oxopropanoate (335 mg, 1.15 mmol) and the corresponding reagents. The crude product was purified by silica gel flash chromatography (eluting with a gradient of 98:2-95:5 methylene chloride/methanol) to yield 197 mg (32%) of ethyl 8-(4-chloro-3-methoxyphenyl)-4-[4-(4-methylpiperazin-1-yl)phenyl]-7-pyridin-4-ylpyrazolo[5,1-c][1,2,4]triazine-3-carboxylate as a red oil, MS 584.4 [M+H].

### Example 26

### 2-Chloro-5-{4-[4-(4-methyl piperazin-1-yl)phenyl]-7-pyridin-4-yl pyrazolo[5,1-c][1,2,4]triazin-8-yl}phenol

The compound 2-chloro-5-{4-[4-(4-methylpiperazin-1-yl)phenyl]-7-pyridin-4-ylpyrazolo[5,1-c][1,2,4]triazin-8-yl} was prepared by the method of example 5, steps 1 and 2, from ethyl 8-(4-chloro-3-methoxyphenyl)-4-[4-(4-methylpiperazin-1-yl)phenyl]-7-pyridin-4-ylpyrazolo[5,1-c][1,2,4]triazine-3-carboxylate. Following silica gel chromatography (gradient 98:2-3:1 methylene chloride/methanol), 2-chloro-5-{4-[4-(4-methylpiperazin-1-yl)phenyl]-7-pyridin-4-ylpyrazolo[5,1-c][1,2,4]triazin-8-yl}phenol was obtained as an orange solid in 44% yield for the 2 steps. MS 498.3 [M+H].

### Example 27

### 8-(4-Chloro-3-methoxyphenyl)-4-phenyl-7-pyridin-4-ylpyrazolo[5,1-c][1,2,4]triazine

To an ice cold (0°-5° C.) mixture of 4-(4-chloro-3-methoxyphenyl)-5-pyridine 4-yl-1H-pyrazol-3-amine (0.05 g, 0.17 mmol) and concentrated hydrochloric acid (0.08 mL) in water (0.17 mL) was added a cold solution of sodium nitrite (16 mg, 0.23 mmol) in water (0.05 mL), and the resulting mixture was stirred for 30 minutes. To this was added methylene chloride (2 mL) and the mixture was basified with saturated solution of sodium carbonate to pH 8-9, then more methylene chloride was added until all the solid was dissolved. Following separation of the layers, the methylene chloride extract was directly added to a cold (0°-5° C.) solution of benzoylmethylene triphenylphosphorane (65.0 mg, 0.17 mmol) in methylene chloride (2 mL) and stirred at 0°-5° C. for 1 hour. The solvent was evaporated and the residue was purified by preparative silica gel chromatography, eluting with 3% methanol in methylene chloride to yield 52 mg (74%) of 8-(4-chloro-3-methoxyphenyl)-4-phenyl-7-pyridin-4-ylpyrazolo[5,1-c][1,2,4]triazine as an orange solid, MS 414.3,[M+H].

### Example 28

### Ethyl 8-(3-methoxyphenyl)-4-[4-(4-methylpiperazin-1-yl)phenyl]-7-pyridin-4-yl pyrazolo[5,1-c][1,2,4]triazine-3-carboxylate

The compound ethyl 8-(3-methoxyphenyl)-4-[4-(4-methylpiperazin-1-yl)phenyl]-7-pyridin-4-ylpyrazolo[5,1-c][1,2,4]triazine-3-carboxylate prepared by the method of example 1 from 4-[3-methoxy-phenyl]-5-pyridin-4-yl-1H-pyrazol-3-amine (279 mg, 1.05 mmol), ethyl 3-[4-(4-methylpiperazin-1-yl)phenyl]-3-oxopropanoate (335 mg, 1.15 mmol) and the corresponding reagents. The crude product was purified by silica gel flash chromatography (eluting with a gradient of 98:2-95:5 methylene chloride/methanol) to yield 201 mg (35%) of ethyl 8-(3-methoxyphenyl)-4-[4-(4-methylpiperazin-1-yl)phenyl]-7-pyridin-4-ylpyrazolo[5,1-c][1,2,4]triazine-3-carboxylate as a red-orange solid, MS 550.4 [M+H].

### Example 29

### 3-{4-[4-(4-Methyl piperazin-1-yl)phenyl]-7-pyridin-4-yl pyrazolo[5,1-c][1,2,4]triazin-8-yl}phenol

The compound 3-{4-[4-(4-methylpiperazin-1-yl)phenyl]-7-pyridin-4-ylpyrazolo[5,1-c][1,2,4]triazin-8-yl}phenol was prepared by the method of example 5, steps 1 and 2, from ethyl 8-(3-methoxyphenyl)-4-[4-(4-methylpiperazin-1-yl)phenyl]-7-pyridin-4-ylpyrazolo[5,1-c][1,2,4]triazine-3-carboxylate. Following silica gel chromatography (gradient 98:2-9:1 methylene chloride/methanol), 3-{4-[4-(4-methylpiperazin-1-yl)phenyl]-7-pyridin-4-ylpyrazolo[5,1-c][1,2,4]triazin-8-yl}phenol was obtained as an orange solid in 57% yield for the 2 steps. MS 464.4 [M+H].

### Example 30

### 3-[8-(4-fluoro-3-hydroxyphenyl)-7-pyridin-4-yl pyrazolo[5,1-c][1,2,4]triazin-4-yl]-N,N-dimethyl benzenesulfonamide, trifluoroacetate salt

### Step 1

### 3-(3-Dimethylsulfamoyl-phenyl)-3-oxo-propionic acid ethyl ester

3-(3-dimethylsulfamoyl-phenyl)-3-oxo-propionic acid ethyl ester was prepared following the procedure described for Example 12, step 1, by the reaction of 3-acetyl-N,N-dimethyl-benzenesulfonamide (0.3 g, 1.32 mmol), with diethyl carbonate (0.35 mL, 2.89 mmol). The crude product was purified by silica gel flash chromatography eluting with 25% ethyl acetate in hexane to yield 0.11g (28%) of 3-(3-dimethylsulfamoyl-phenyl)-3-oxo-propionic acid ethyl ester as a colorless oil, MS 300.3 [M+H].

### Step 2

### 4-(3-Dimethylsulfamoyl-phenyl)-8-(4-fluoro-3-hydroxy-phenyl)-7-pyridin-4-yl-pyrazolo[5,1-c][1,2,4]triazine-3-carboxylic acid ethyl ester

4-(3-Dimethylsulfamoyl-phenyl)-8-(4-fluoro-3-hydroxy-phenyl)-7-pyridin-4-yl-pyrazolo[5,1-c][1,2,4]triazine-3-carboxylic acid ethyl ester was prepared by the method of example 1 from 4-(4-fluoro-3-methoxyphenyl)-3-pyridin-4-yl-1H-pyrazol-5-amine (0.105 g, 0.37 mmol), 3-(3-dimethylsulfamoyl-phenyl)-3-oxo-propionic acid ethyl ester (0.11g, 0.37 mmol) and the corresponding reagents. The crude product was purified by silica gel flash chromatography, eluting with 3% methanol in methylene chloride to yield 84.0 mg (39%) of 4-(3-dimethylsulfamoyl-phenyl)-8-(4-fluoro-3-hydroxy-phenyl)-7-pyridin-4-yl-pyrazolo[5,1-c][1,2,4]triazine-3-carboxylic acid ethyl ester as a yellow solid, MS 577.2 [M+H].

### Step 3

### 4-(3-Dimethylsulfamoyl-phenyl)-8-(4-fluoro-3-hydroxy-phenyl)-7-pyridin-4-yl-pyrazolo[5,1-c][1,2,4]triazine-3-carboxylic acid

4-(3-Dimethylsulfamoyl-phenyl)-8-(4-fluoro-3-hydroxy-phenyl)-7-pyridin-4-yl-pyrazolo[5,1-c][1,2,4]triazine-3-carboxylic acid was prepared following the method for example 2, step 1, using 4-(3-dimethylsulfamoyl-phenyl)-8-(4-fluoro-3-hydroxy-phenyl)-7-pyridin-4-yl-pyrazolo[5,1-c][1,2,4]triazine-3-carboxylic acid ethyl ester (0.08 g, 0.14 mmol) and boron tribromide (1 M solution in methylene chloride, 3.5 ml, 3.5 mmol). The crude product was used in the next step without further purification.

### Step 4

### 3-[8-(4-fluoro-3-hydroxyphenyl)-7-pyridin-4-yl pyrazolo[5,1-c][1,2,4]triazin-4-yl]-N,N-dimethyl benzenesulfonamide, trifiuoroacetate salt

3-[8-(4-Fluoro-3-hydroxyphenyl)-7-pyridin-4-ylpyrazolo[5,1-c][1,2,4]triazin-4-yl]-N,N-dimethylbenzenesulfonamide was prepared following the above method for example 5, step 2 using crude 4-(3-dimethylsulfamoyl-phenyl)-8-(4-fluoro-3-hydroxy-phenyl)-7-pyridin-4-yl-pyrazolo[5,1-c][1,2,4]triazine-3-carboxylic acid (0.06 g) in Dowtherm (1.0 mL) at 165° C. for 3 minutes. The crude product was purified by silica gel flash chromatography, eluting with 2% methanol in methylene chloride, and then with reverse phase HPLC to yield 23 mg of 3-[8-(4-fluoro-3-hydroxyphenyl)-7-pyridin-4-ylpyrazolo[5,1-c][1,2,4]triazin-4-yl]-N,N-dimethylbenzenesulfonamide (trifluoroacetate salt) as a yellow solid. MS 491.1,[M+H].

What is claimed is:

1. A compound of formula A: 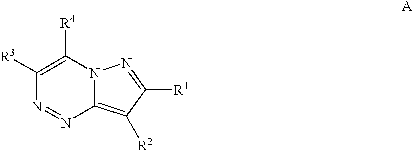
and pharmaceutically acceptable salts thereof;
wherein R¹ is a 5-7 membered heterocyclic ring or a heteroaryl ring containing 1-3 heteroatoms selected from N, O or S, or an aryl ring, each ring substituted with one to four substituents selected from the group consisting of: —F, —Cl, Br, —I, —NO₂, —CN, —N₃, —CHO, —CF₃, —OCF₃, —R⁵, —OR⁵, —S(O)ₘR⁵, —S(O)ₘNR⁵R⁵, —NR⁵R⁵, —NR⁵S(O)ₘR⁵, —OR⁷OR⁵, —OR⁷NR⁵R⁵, —N(R⁵)R⁷OR⁵, —N(R⁵)R⁷NR⁵R⁵, —NR⁵C(O)R⁵, —C(O)R⁵, —C(O)OR⁵, —C(O)NR⁵R⁵, —OC(O)R⁵, —OC(O)OR⁵, —OC(O)NR⁵R⁵, NR⁵C(O)R⁵, —NR⁵C(O)OR⁵, —NR⁵C(O)NR⁵R⁵, —R⁶OR⁵, —R⁶OR⁷OR⁵, —R⁶OR⁷NR⁵R⁵, —R⁶N(R⁵)R⁷OR⁵, —R⁶N(R⁵)R⁷NR⁵R⁵, —R⁶NR⁵R⁵, —R⁶S(O)ₘR⁵, —R⁶S(O)ₘNR⁵R⁵, —R⁶C(O)R⁵, —R⁶C(O)R⁵, —R⁶C(O)NR⁵R⁵, —R⁶OC(O)R⁵, —R⁶OC(O)OR⁵, —R⁶NR⁵S(O)ₘR⁵, —R⁶OC(O)NR⁵R⁵, —R⁶NR⁵C(O)R⁵, —R⁶NR⁵C(O)OR⁵ or —R⁶NR⁵C(O)NR⁵R⁵;
R² is an aryl ring substituted with at least one substituent —OR⁸, up to four other substituents, each other substituent independently selected from the group consisting of: —F, —Cl, Br, —I, —NO₂, —CN, —N3, —CHO, —CF₃, —OCF₃, —R⁵, —OR⁵, —S(O)ₘR⁵, —NR⁵R⁵, —NR⁵S(O)ₘR⁵, —S(O)ₘNR⁵R⁵, —OR⁷OR⁵, —OR⁷NR⁵R⁵, —N(R⁵)R⁷OR⁵, —N(R⁵)R⁷NR⁵R⁵, —NR⁵C(O)R⁵, —C(O)R⁵, —C(O)OR⁵, —C(O)NR⁵R⁵, —OC(O)R⁵, —OC(O)OR⁵, —OC(O)NR⁵R⁵, NR⁵C(O)R⁵, —NR⁵C(O)OR⁵, —NR⁵C(O)NR⁵R⁵, —R⁶OR⁵, —R⁶OR⁷OR⁵, —R⁶OR⁷NR⁵R⁵, —R⁶N(R⁵)R⁷OR⁵, —R⁶N(R⁵)R⁷NR⁵R⁵, —R⁶NR⁵R⁵, —R⁶S(O)ₘR⁵, —R⁶NR⁵S(O)ₘR⁵, —R⁶S(O)ₘNR⁵R⁵, —R⁶C(O)R⁵, —R⁶C(O)OR⁵, —R⁶C(O)NR⁵R⁵, —R⁶OC(O)R⁵, —R⁶OC(O)OR⁵, —R⁶OC(O)NR⁵R⁵, —R⁶NR⁵C(O)R⁵, —R⁶NR⁵C(O)OR⁵ or —R⁶NR⁵C(O)NR⁵R⁵;
R³ and R⁴ are independently selected from the group consisting of: H, cycloalkyl of 3-10 carbons, alkyl of 1-6 carbons, alkoxy of 1-6 carbons, cycloalkoxy of 3-10 carbons, alkene of 1-6 carbons, alkyne of 1-6 carbons; aryl ring, heterocyclic ring and heteroaryl ring containing 1-3 heteroatoms selected from N, O or S; each ring substituted with one to four substituents selected from the group consisting of: —N₃, —CHO, —OCF₃, —S(O)ₘR⁸, —NR⁸R⁸, —NR⁸S(O)ₘR⁸, —S(O)ₘNR⁸R⁸, —OR⁷OR⁸, —OR⁷NR⁸R⁸, —N(R⁸)R⁷OR⁸, —N(R⁸)R⁷NR⁸R⁸, —NR⁸C(O)R⁸, —C(O)R⁸, —C(O)OR⁸, —C(O)NR⁸R⁸, —OC(O)R⁸, —OC(O)OR⁸, —OC(O)NR⁸R⁸, NR⁸C(O)R⁸, —NR⁸C(O)OR⁸, —NR⁸C(O)NR⁸R⁸, —R⁶OR⁸, —R⁶NR⁸R⁸, —R⁶S(O)ₘR⁸, —R⁶NR⁸S(O)ₘR⁸, —R⁶S(O)ₘNR⁸R⁸, —R⁶C(O)R⁸,—R⁶C(O)OR⁸, —R⁶OR⁷OR⁸, —R⁶OR⁷NR⁸R⁸, —R⁶N(R⁸)R⁷OR⁸, —R⁶N(R⁸)R⁷NR⁸R⁸, —R⁶C(O)NR⁸R⁸, —R⁶OC(O)R⁸, —R⁶OC(O)OR⁸, —R⁶OC(O)NR⁸R⁸, —R⁶NR⁸C(O)R⁸, —R⁶NR⁸C(O)OR⁸, —R⁶NR⁸C(O)NR⁸R⁸ or —YR⁸;
R⁵ is selected from the group consisting of: H, alkyl of 1-6 carbon atoms, branched alkyl of 1-8 carbon atoms, alkenyl of 2-6 carbon atoms, alkynyl of 2-6 carbon atoms or cycloalkyl of 3-7 carbons;
R⁶ is a divalent group selected from the group consisting of: alkyl of 1-6 carbon atoms, alkenyl of 2-6 carbon atoms, and alkynyl of 2-6 carbon atoms;
R⁷ is a divalent alkyl group of 2-6 carbon atoms;
R⁸ is selected from the group consisting of: H, alkyl of 1-6 carbon atoms, alkenyl of 2-6 carbon atoms, alkynyl of 2-6 carbon atoms, aryl of 6-12 carbons, substituted aryl of 6-12 carbons, substituted heterocyclyl ring and substituted heteroaryl ring containing 1-3 heteroatoms selected from N, O or S;
m is an integer of 0-2; and
Y is —N(R⁵)—, —N(R⁵)R⁶—, —O—, —OR⁶—, —S(O)ₘ— or —S(O)ₘR⁶—.

2. The compound of claim 1, wherein R² is a mono- or a di-substituted monocyclic aryl ring.

3. The compound of claim 1, wherein R² is phenol.

4. The compound of claim 1, wherein R² is phenol substituted further with a substituent selected from the group consisting of: —F, —Cl, Br, —I, —NO₂, —CN, —N₃, —CHO, —CF₃, —OCF₃, —R⁵, —OR⁵, —S(O)ₘR⁵, and —NR⁵R⁵.

5. The compound of claim 1, wherein R² is C1-C6 alkoxyphenyl.

6. The compound of claim 1, wherein R² is methoxyphenyl substituted further with a substituent selected from the group consisting of: —F, —Cl, Br, —I, —NO₂, —CN, —N₃, —CHO, —CF₃, —OCF₃, —R⁵, —OR⁵, —S(O)ₘR⁵, and —NR⁵R⁵.

7. A compound of formula A: 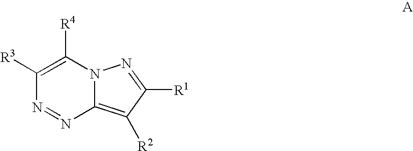
and pharmaceutically acceptable salts thereof;
wherein R¹ is a 5-7 membered heterocyclic ring or a heteroaryl ring containing 1-3 heteroatoms selected from N, O or S, or an aryl ring, each ring substituted with one to four substituents selected from the group consisting of: —F, —Cl, Br, —I, —NO₂, —CN, —N₃, —CHO, —CF₃, —OCF₃, —R⁵, —OR⁵, —S(O)ₘR⁵, —S(O)ₘNR⁵R⁵, —NR⁵R⁵, —NR⁵S(O)ₘR⁵, —OR⁷OR⁵, —OR⁷NR⁵R⁵, —N(R⁵)R⁷OR⁵, —N(R⁵)R⁷NR⁵R⁵, —NR⁵C(O)R⁵, —C(O)R⁵, —C(O)OR⁵, —C(O)NR⁵R⁵, —OC(O)R⁵, —OC(O)OR⁵, —OC(O)NR⁵R⁵, NR⁵C(O)R⁵, —NR⁵C(O)OR⁵, —NR⁵C(O)NR⁵R⁵, —R⁶OR⁵, —R⁶OR⁷OR⁵, —R⁶OR⁷NR⁵R⁵, —R⁶N(R⁵)R⁷OR⁵, —R⁶N(R⁵)R⁷NR⁵R⁵, —R⁶NR⁵R⁵, —R⁶S(O)ₘR⁵, —R⁶S(O)ₘNR⁵R⁵, —R⁶C(O)R⁵, —R⁶C(O)R⁵, —R⁶C(O)NR⁵R⁵, —R⁶OC(O)R⁵, —R⁶OC(O)OR⁵, —R⁶NR⁵S(O)ₘR⁵, —R⁶OC(O)NR⁵R⁵, R⁶NR⁵C(O)R⁵, —R⁶NR⁵C(O)OR⁵ or —R⁶NR⁵C(O)NR⁵R⁵;
R² is a bicyclic heteroaryl ring of formula 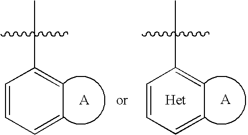
wherein 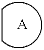
is a 5-7 membered heteroaryl ring containing 1-3 heteroatoms selected from N, or S,
Het is a 6-membered heteroaryl ring containing 1-2 nitrogen atoms, and either bicyclic heteroaryl ring is substituted with one to four substituents, each substituent independently selected from the group consisting of: —F, —Cl, Br, —I, —NO₂, —CN, —N₃, —CHO, —CF₃, —OCF₃, —R⁵, —OR⁵, —S(O)ₘR⁵, —NR⁵R⁵;
R³ and R⁴ are independently selected from the group consisting of: H, cycloalkyl of 3-10 carbons, alkyl of 1-6 carbons, alkoxy of 1-6 carbons, cycloalkoxy of 3-10 carbons, alkene of 1-6 carbons, alkyne of 1-6 carbons; aryl ring, heterocyclic ring and heteroaryl ring containing 1-3 heteroatoms selected from N, O or S; each ring substituted with one to four substituents selected from the group consisting of: —N₃, —CHO, —OCF₃, —S(O)ₘR⁸, —NR⁸R⁸, —NR⁸S(O)ₘR⁸, —S(O)ₘNR⁸R⁸, —OR⁷OR⁸, —OR⁷NR⁸R⁸, —N(R⁸)R⁷OR⁸, —N(R⁸)R⁷NR⁸R⁸, —NR⁸C(O)R⁸, —C(O)R⁸, —C(O)OR⁸, —C(O)NR⁸R⁸, OC(O)R⁸, —OC(O)OR⁸, —OC(O)NR⁸R⁸, NR⁸C(O)R⁸, —NR⁸C(O)OR⁸, —NR⁸C(O)NR⁸R⁸, —R⁶OR⁸, —R⁶NR⁸R⁸, —R⁶S(O)ₘR⁸, —R⁶NR⁸S(O)ₘR⁸, —R⁶S(O)ₘNR⁸R⁸, —R⁶C(O)R⁸, —R⁶C(O)OR⁸, —R⁶OR⁷OR⁸, —R⁶OR⁷NR⁸R⁸, —R⁶N(R⁸)R⁷OR⁸, —R⁶N(R⁸)R⁷NR⁸R⁸, —R⁶C(O)NR⁸R⁸, —R⁶OC(O)R⁸, —R⁶OC(O)OR⁸, —R⁶OC(O)NR⁸R⁸, —R⁶NR⁸C(O)R⁸, —R⁶NR⁸C(O)OR⁸, —R⁶NR⁸C(O)NR⁸R⁸ or —YR⁸;
R⁵ is selected from the group consisting of: H, alkyl of 1-6 carbon atoms, branched alkyl of 1-8 carbon atoms, alkenyl of 2-6 carbon atoms, alkynyl of 2-6 carbon atoms or cycloalkyl of 3-7 carbons;
R⁶ is a divalent group selected from the group consisting of: alkyl of 1-6 carbon atoms, alkenyl of 2-6 carbon atoms, and alkynyl of 2-6 carbon atoms;
R⁷ is a divalent alkyl group of 2-6 carbon atoms;
R⁸ is selected from the group consisting of: H, alkyl of 1-6 carbon atoms, alkenyl of 2-6 carbon atoms, alkynyl of 2-6 carbon atoms, aryl of 6-12 carbons, substituted aryl of 6-12 carbons, substituted heterocyclyl ring and substituted heteroaryl ring containing 1-3 heteroatoms selected from N, O or S;
m is an integer of 0-2; and
Y is —N(R⁵)—, —N(R⁵)R⁶—, —O—, —OR⁶—, —S(O)ₘ— or —S(O)ₘR⁶—.

8. The compound of claim 7, wherein R² is selected from the group consisting of:
indolyl, benzimidazolyl and indazolidyl.

9. A method for making a compound of formula A: 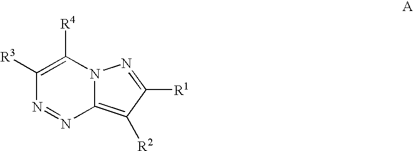
and pharmaceutically acceptable sats thereof; comprising the steps of: (a) reacting a substituted aminopyrazole of formula 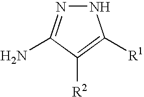
with a mixture of sodium nitrate and a strong acid in an aqueous solvent; and (b) adding a substituted β-keto acid or a substituted β-keto ester of formula: 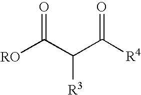
and a base to the mixture,
wherein R is H or C₁-C₆ alkyl, R¹ is a 5-7 membered heterocyclic ring or a heteroaryl ring containing 1-3 heteroatoms selected from N, O or S, or a monocyclic aryl ring, each ring substituted with one to four substituents selected from the group consisting of: —F, —Cl, Br, —I, —NO₂, —CN, —N₃, —CHO, —CF₃, —OCF₃, —R⁵, —OR⁵, —S(O)ₘR⁵, S(O)ₘNR⁵R⁵, —NR⁵R⁵, —NR⁵S(O)ₘR⁵, —OR⁷OR⁵, —OR⁷NR⁵R⁵, —N(R⁵)R⁷OR⁵, —N(R⁵)R⁷NR⁵R⁵, —NR⁵C(O)R⁵, —C(O)R⁵, —C(O)OR⁵, —C(O)NR⁵R⁵, —OC(O)R⁵, —OC(O)OR⁵, —OC(O)NR⁵R⁵, NR⁵C(O)R⁵, —NR⁵C(O)OR⁵, —NR⁵C(O)NR⁵R⁵, —R⁶OR⁵, —R⁶OR⁷OR⁵, —R⁶OR⁷NR⁵R⁵, —R⁶N(R⁵)R⁷OR⁵, —R⁶N(R⁵)R⁷NR⁵R⁵, —R⁶NR⁵R⁵, —R⁶S(O)ₘR⁵, —R⁶S(O)ₘNR⁵R⁵, —R⁶C(O)R⁵, —R⁶C(O)OR⁵, —R⁶C(O)NR⁵R⁵, —R⁶OC(O)R⁵, —R⁶OC(O)OR⁵, —R⁶NR⁵S(O)ₘR⁵, —R⁶OC(O)NR⁵R⁵, —R⁶NR⁵C(O)R⁵, —R⁶NR⁵C(O)OR⁵ or —R⁶NR⁵C(O)NR⁵R⁵;
R² is a monocyclic aryl ring substituted with at least one substituent —OR⁸, up to four other substituents, each other substituent independently selected from the group consisting of: —F, —Cl, Br, —I, —NO₂, —CN, —N₃, —CHO, —CF₃, —OCF₃, —R⁵, —OR⁵, —S(O)ₘR⁵, —NR⁵R⁵, —NR⁵S(O)ₘR⁵, —S(O)ₘNR⁵R⁵, —OR⁷OR⁵, —OR⁷NR⁵R⁵, —N(R⁵)R⁷OR⁵, —N(R⁵)R⁷NR⁵R⁵, —NR⁵C(O)R⁵, —C(O)R⁵, —C(O)OR⁵, —C(O)NR⁵R⁵, —OC(O)R⁵, —OC(O)OR⁵, —OC(O)NR⁵R⁵, NR⁵C(O)R⁵, —NR⁵C(O)OR⁵, —NR⁵C(O)NR⁵R⁵, —R⁶OR⁵, —R⁶OR⁷OR⁵, —R⁶OR⁷NR⁵R⁵, —R⁶N(R⁵)R⁷OR⁵, —R⁶N(R⁵)R⁷NR⁵R⁵, —R⁶NR⁵R⁵, —R⁶S(O)ₘR⁵, —R⁶NR⁵S(O)ₘR⁵, —R⁶S(O)ₘNR⁵R⁵, —R⁶C(O)R⁵, —R⁶C(O)OR⁵, —R⁶C(O)NR⁵R⁵, —R⁶OC(O)R⁵, —R⁶OC(O)OR⁵, —R⁶OC(O)NR⁵R⁵, —R⁶NR⁵C(O)R⁵, —R⁶NR⁵C(O)OR⁵ or —R⁶NR⁵C(O)NR⁵R⁵ or R² is a bridged bicyclic heteroaryl ring of formula 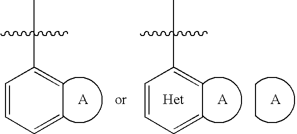
is a 5-7 membered heteroaryl ring containing 1-3 heteroatoms selected from N, O or S,
Het is a 6-membered heteroaryl ring containing 1-2 nitrogen atoms, and either bridged bicyclic heteroaryl ring is substituted with one to four substituents, each substituent independently selected from the group consisting of: —F, —Cl, Br, —I, —NO₂, —CN, —N₃, —CHO, —CF₃, —OCF₃, —R⁵, —OR⁵, —S(O)ₘR⁵, —NR⁵R⁵;
R³ and R⁴ are independently selected from the group consisting of: H, cycloalkyl of 3-10 carbons, alkyl of 1-6 carbons, alkoxy of 1-6 carbons, cycloalkoxy of 3-10 carbons, alkene of 1-6 carbons, alkyne of 1-6 carbons; aryl ring, heterocyclic ring and heteroaryl ring containing 1-3 heteroatoms selected from N, O or S; each ring substituted with one to four substituents selected from the group consisting of: —F, —Cl, Br, —I, —NO₂, —CN, —N₃, —CHO, —OCF₃, —S(O)ₘR⁸, —NR⁸R⁸, —NR⁸S(O)ₘR⁸, —S(O)ₘNR⁸R⁸, —OR⁷OR⁸, —OR⁷NR⁸R⁸, —N(R⁸)R⁷OR⁸, —N(R⁸)R⁷NR⁸R⁸, —NR⁸C(O)R⁸, —C(O)R⁸, —C(O)OR⁸, —C(O)NR⁸R⁸, —OC(O)R⁸, —OC(O)OR⁸, —OC(O)NR⁸R⁸, NR⁸C(O)R⁸, —NR⁸C(O)OR⁸, —NR⁸C(O)NR⁸R⁸, —R⁶OR⁸, —R⁶NR⁸R⁸, —R⁶S(O)ₘR⁸, —R⁶NR⁸S(O)ₘR⁸, —R⁶S(O)ₘNR⁸R⁸, —R⁶C(O)R⁸, —R⁶C(O)OR⁸, —R⁶OR⁷OR⁸, —R⁶OR⁷NR⁸R⁸, —R⁶N(R⁸)R⁷OR⁸, —R⁶N(R⁸)R⁷NR⁸R⁸, —R⁶C(O)NR⁸R⁸, —R⁶OC(O)R⁸, —R⁶OC(O)OR⁸, —R⁶OC(O)NR⁸R⁸, —R⁶NR⁸C(O)R⁸, —R⁶NR⁸C(O)OR⁸, —R⁶NR⁸C(O)NR⁸R⁸ or —YR⁸;
R⁵ is selected from the group consisting of: H, alkyl of 1-6 carbon atoms, branched alkyl of 1-8 carbon atoms, alkenyl of 2-6 carbon atoms, alkynyl of 2-6 carbon atoms or cycloalkyl of 3-7 carbons;
R⁶ is a divalent group selected from the group consisting of: alkyl of 1-6 carbon atoms, alkenyl of 2-6 carbon atoms, and alkynyl of 2-6 carbon atoms;
R⁷ is a divalent alkyl group of 2-6 carbon atoms;
R⁸ is selected from the group consisting of: H, alkyl of 1-6 carbon atoms, alkenyl of 2-6 carbon atoms, alkynyl of 2-6 carbon atoms, aryl of 6-12 carbons, substituted aryl of 6-12 carbons, substituted heterocyclyl ring and substituted heteroaryl ring containing 1-3 heteroatoms selected from N, O or S;
m is an integer of 0-2; and
Y is —N(R⁵)—, —N(R⁵)R⁶—, —O—, —OR⁶—, —S(O)ₘ— or —S(O)ₘR⁶—.

10. The method according to claim 9 wherein in step (b) a substituted β-keto ester is added of formula: 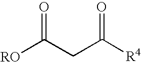
and further comprises (c) hydrolyzng the ester formed of formula: 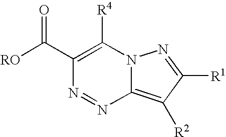
to a corresponding carboxylic acid using an aqueous acid or to a corresponding carboxylate salt using an aqueous base; and (d) heating the compound of formula: 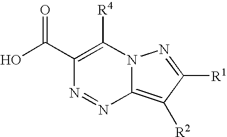
to a temperature of 150° C. or higher.

11. The method according to claim 9 wherein in step (b) a substituted β-keto ester is added of formula: 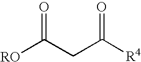
and further comprises (c) hydrolyzng the ester formed of formula: 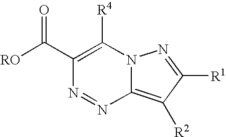
to a corresponding carboxylic acid compound of formula: 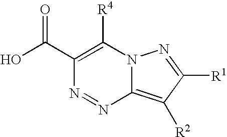
and (d) reacting the carboxylic acid compound with substituted amines.

12. The method according to claim 9 wherein in step (b) a substituted β-keto sulfone is added of formula: 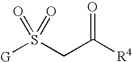
and a base to the mixture; and (c) reducing the sulfone formed of formula: 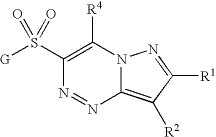
with a reducing agent, wherein G is selected from the group consisting of: alkyl of 1-6 carbon atoms, an aryl of 6-12 carbons and a substituted aryl of 6-12 carbons.

13. A pharmaceutical composition comprising a compound according to any of claims 1-8 and a pharmaceutically acceptable carrier.

14. A pharmaceutical composition comprising a compound according to any of claims 1-8 in combination with other kinase-inhibiting pharmaceutical compositions or chemotherapeutic agents, and a pharmaceutically acceptable carrier.

15. A method of inhibiting kinase activity in a mammal comprising administering to a mammal a kinase-inhibiting amount of a compound according to any one of claims 1-8.

16. The method of claim 15, wherein the mammal is a human.

17. A method of treating a kinase-dependent condition comprising administering to a subject a kinase-inhibiting amount of a compound according to any one of claims 1-8.

18. A method of treating a B-Raf kinase-dependent condition comprising inflammation or cancer, by administering to a patient a compound any one of claims 1-8.

19. The method of claim 18, wherein the cancer is cancer is selected from the group consisting of: breast, kidney, bladder, mouth, larynx, esophagus, stomach, colon, ovary, lung, pancreas, skin, liver, prostate and brain cancer.
